(19) United States
(12) Patent Application Publication
(10) Pub. No.: US 2014/0255930 A1
(43) Pub. Date: Sep. 11, 2014

(54) Materials and Methods Related to Dopamine Dysregulation Disorders

(71) Applicants: Wolfgang Sadee, Upper Arlington, OH (US); Danielle Sullivan, Chicago, IL (US); Julia Pinsonneault, Columbus, OH (US)

(72) Inventors: Wolfgang Sadee, Upper Arlington, OH (US); Danielle Sullivan, Chicago, IL (US); Julia Pinsonneault, Columbus, OH (US)

(73) Assignee: Ohio State Innovation Foundation, Columbus, OH (US)

(21) Appl. No.: 14/237,038

(22) PCT Filed: Sep. 10, 2012
(86) PCT No.: PCT/US2012/054473

§ 371 Date: May 16, 2014

Related U.S. Application Data
(60) Provisional application No. 61/532,281, filed on Sep. 8, 2011.

(51) Int. Cl. C12Q 1/68

(52) U.S. Cl. CPC C12Q 1/6883
USPC 435/6.11

(57) ABSTRACT

The present invention provides methods of identifying an increased risk of a dopamine dysregulation disorder in a human subject, including identifying increased risk of dementia, Parkinson's disease, Huntington's, epilepsy, mania, attention deficit disorder, anxiety, dyslexia, schizophrenia, depression, obsessive-compulsive disorders, alcoholism, obesity, addiction disorders, pathological gambling, attention deficit hyperactivity disorder, bipolar disorder, Tourette syndrome, substance dependence, sub stance abuse, substance overdose, and substance-related death.

### CROSS REFERENCE TO RELATED APPLICATIONS

This application claims the benefit of U.S. Provisional Application No. 61/532,281 filed Sep. 8, 2011, the disclosure of which is incorporated herein by reference for all purposes.

### STATEMENT REGARDING FEDERALLY SPONSORED RESEARCH

This invention was made with government support (under National Institute on Drug Abuse grant R01 DA022199 and U01 GM092655). The government may have certain rights in this invention.

### TECHNICAL FIELD OF THE INVENTION

The present invention relates to regulatory polymorphisms in dopamine D2 receptor (DRD2) and transporter (DAT). Dysregulation of DRD2 and/or DAT can affect the risk for psychiatric disorders. Therefore, the present invention relates generally to the fields of medicine, biochemistry, psychology, and statistics.

### BACKGROUND OF THE INVENTION

Genetic factors contribute to complex human traits and diseases, but the extent and nature of genetic influence often remains uncertain. Whether frequent or rare genetic variants in few or many genes are responsible remains a matter of debate. Gene-gene and gene-environment interactions likely play a critical role, but few studies show how two or more genes combined have strong impact on a complex trait, or when this interaction is contingent upon external conditions. In C. elegans, genetic variation in catechol receptors and environmental cues converge to regulate behavioral decisions, suggesting an ancient role and evolution of catecholamine signaling in behavior and providing a rare example of a gene-gene-environment interaction (npr-1/tyra-3/nutrient supply). In humans, a number of genes involved in behavioral modulation carry frequent variants, often regulating gene expression and mRNA processing or translation, possibly as a result of evolutionary selection pressures. Here the inventors present a gene-gene-environment interaction (cocaine exposure) affecting risk of severe cocaine abuse and death.

Genetic factors contribute strongly to drug addiction but remain only partially understood. While each class of addictive drugs taps into different neuronal and biochemical targets, downstream processes reliably include dopaminergic signaling. Cocaine and similar stimulant drugs target neurotransmitter transporter, and in particular the dopamine transporter DAT (SLC6A3) Inhibition of DAT leads to elevated synaptic dopamine levels—a contributor to the ‘high’ experienced upon cocaine ingestion. Another critical player in dopaminergic signaling, the dopamine D2 receptor (encoded by DRD2) occurs in two main splice variants, a short form lacking exon6 (D2S) and a long form with exon6 (D2L). Being largely expressed presynaptically, D2S is considered an autoreceptor inhibiting dopamine release upon activation. Importantly, D2S and DAT have been shown to physically interact with each other, with D2S facilitating DAT functional expression in the presynaptic membrane.

### SUMMARY OF THE INVENTION

Aberrant dopaminergic signaling plays a pervasive role in CNS disorders, with DRD2 and DAT being central factors. Among several regulatory polymorphisms, DRD2 SNP rs2283265 reduces formation of the short D2 splice isoform D2S, conveying significant risk (odds ratio OR ˜3) of severe cocaine abuse and death in subjects obtained from the Miami Dowd County Brain Endowment Bank. Similarly for the main cocaine target DAT, frequent regulatory variants have been identified including rs3836790 (intron8 5/6-repeat) and rs27072 in the 3′UTR 2. Because DAT and D2S physically and functionally interact in presynaptic membranes, the inventors investigated DAT and DRD2 gene interactions in the Miami cohort. Whereas the DAT variants alone were not significantly associated with cocaine abuse/death, strong interactions were observed between DRD2 rs2283265 and DAT intron8 5/6-repeat. The minor 5-repeat DAT allele (˜33% allele frequency (MAF) in Caucasians) protected against risk conferred by the minor rs2283265 allele. On the other hand, the calculated risk of rs2283265 increased to 7.6 OR (CI: 2.3-25, p=0.0002) in the absence of the minor 5-repeat DAT allele. High frequency of the protective DAT intron8 5-repeat allele in African Americans (65%) accounts for a reported lack of detectable risk conferred by rs2283265 in this ethnic group (Moyer et al., 2011) Similarly, DRD2 rs2283265 and DAT intron8 5/6-repeat interacted to affect DAT protein expression in prefrontal cortex of cocaine abusers. Lastly, a rare haplotype consisting of the minor alleles of DAT intron8 and rs27072 occurred at high frequency in African American cocaine abusers (˜17%) but was absent in the controls, implying high ORs. These results demonstrate strong gene-gene- and haplotype-environment effects, associated with a substantially increased risk of cocaine abuse and overdose.

Therefore, embodiments of the present invention provide methods of identifying an increased risk of a dopamine dysregulation disorder in a human subject comprising:
a.) determining, in a nucleic acid-containing sample from a human subject:
   i. the genotype of the DRD2 gene at rs2283265; and
   ii. the number of intron8 repeats in the DAT gene;
b.) identifying increased risk of a dopamine dysregulation disorder if:
   i. the genotype of the DRD2 gene at rs2283265 is heterozygous G/T or homozygous T/T and the number of intron8 repeats in the DAT gene is homozygous 6/6; or
   ii. the genotype of the DRD2 gene at rs2283265 is homozygous G/G and the number of intron8 repeats in the DAT gene is homozygous 5/5 or heterozygous 5/6.

Embodiments of the present invention also provide methods of identifying a decreased risk of a dopamine dysregulation disorder in a human subject comprising:
a.) determining, in a nucleic acid-containing sample from a human subject:
   i. the genotype of the DRD2 gene at rs2283265; and
   ii. the number of intron8 repeats in the DAT gene;
b.) identifying decreased risk of a dopamine dysregulation disorder if:
   i. the genotype of the DRD2 gene at rs2283265 is homozygous T/T and the number of intron8 repeats in the DAT gene is homozygous 5/5 or heterozygous 5/6.

Embodiments of the present invention also provide methods of identifying a decreased risk of a dopamine dysregulation disorder in a human subject comprising:
a.) determining, in a nucleic acid-containing sample from a human subject:
   i. the genotype of the DRD2 gene at rs2283265; and
   ii. the number of intron8 repeats in the DAT gene;
b.) identifying decreased risk of a dopamine dysregulation disorder if:
   i. the genotype of the DRD2 gene at rs2283265 is heterozygous G/T and the number of intron8 repeats in the DAT gene is homozygous 5/5 or heterozygous 5/6; or
   ii. the genotype of the DRD2 gene at rs2283265 is homozygous G/G and the number of intron8 repeats in the DAT gene is homozygous 6/6.

Embodiments of the present invention also provide methods as described, which further comprise determining the genotype of the DRD2 gene at rs1076560.

Embodiments of the present invention also provide methods of identifying an increased risk of a dopamine dysregulation disorder in a Caucasian or Hispanic human subject comprising:
a.) determining, in a nucleic acid-containing sample from a Caucasian or Hispanic human subject:
   i. the genotype of the DRD2 gene at at least one locus selected from the group consisting of rs1076560 and rs2283265; and
b.) identifying increased risk of a dopamine dysregulation disorder if:
   i. the genotype of the DRD2 gene at rs1076560 and/or rs2283265 is heterozygous G/T or homozygous T/T.

Embodiments of the present invention also provide methods of identifying an increased risk of a dopamine dysregulation disorder in a human subject comprising:
a.) determining, in a nucleic acid-containing sample from a human subject:
   i. the genotype of the DRD2 gene at rs1076560 and rs2283265; and
b.) identifying increased risk of a dopamine dysregulation disorder if:
   i. the genotype of the DRD2 gene at rs1076560 and rs2283265 is heterozygous G/T or homozygous T/T.

Embodiments of the present invention also provide methods as described, which further comprise determining the genotype of the DAT gene at least one locus selected from the group consisting of: rs6347; rs27072; and rs3836790.

Embodiments of the present invention also provide methods as described, further comprising recommending a health or legal strategy based on the results of step (b).

Embodiments of the present invention also provide methods as described, wherein determining comprises nucleic acid amplification.

Embodiments of the present invention also provide methods as described, wherein amplification comprises PCR.

Embodiments of the present invention also provide methods as described, wherein determining comprises primer extension.

Embodiments of the present invention also provide methods as described, wherein determining comprises restriction digestion.

Embodiments of the present invention also provide methods as described, wherein determining comprises sequencing.

Embodiments of the present invention also provide methods as described, wherein determining comprises SNP specific oligonucleotide hybridization.

Embodiments of the present invention also provide methods as described, wherein determining comprises a DNAse protection assay.

Embodiments of the present invention also provide methods as described, wherein said nucleic acid-containing sample is blood, sputum, saliva, mucosal scraping or tissue biopsy.

Embodiments of the present invention also provide methods as described, wherein the dopamine dysregulation disorder is selected from the group consisting of: pre-senile dementia (early-onset Alzheimer's disease), senile dementia (dementia of the Alzheimer's type), micro-infarct dementia, AIDS-related dementia, vascular dementia, Parkinsonism including Parkinson's disease, Lewy body dementia, progressive supranuclear palsy, Huntington's chorea, tardive dyskinesia, hyperkinesia, epilepsy, mania, attention deficit disorder, anxiety, dyslexia, schizophrenia, depression, obsessive-compulsive disorders, alcoholism, obesity, pathological gambling, attention deficit hyperactivity disorder, Tourette syndrome, cocaine dependence, nicotine dependence, polysubstance abuse, methamphetamine abuse, morphine abuse, morphine-analogue abuse, prescription drug abuse, illegal drug abuse, and addiction disorders.

Embodiments of the present invention also provide methods of identifying an increased risk of cocaine abuse or overdose in a human subject comprising:
a.) determining, in a nucleic acid-containing sample from a human subject:
   i. the genotype of the DRD2 gene at rs2283265; and
   ii. the number of intron8 repeats in the DAT gene;
b.) identifying increased risk of cocaine abuse or overdose if:
   i. the genotype of the DRD2 gene at rs2283265 is heterozygous G/T or homozygous T/T and the number of intron8 repeats in the DAT gene is homozygous 6/6; or
   ii. the genotype of the DRD2 gene at rs2283265 is homozygous G/G and the number of intron8 repeats in the DAT gene is homozygous 5/5 or heterozygous 5/6.

Embodiments of the present invention also provide methods of identifying a decreased risk of cocaine abuse or overdose in a human subject comprising:
a.) determining, in a nucleic acid-containing sample from a human subject:
   i. the genotype of the DRD2 gene at rs2283265; and
   ii. the number of intron8 repeats in the DAT gene;
b.) identifying decreased risk of developing a dopamine dysregulation disorder if:
   i. the genotype of the DRD2 gene at rs2283265 is homozygous T/T and the number of intron8 repeats in the DAT gene is homozygous 5/5 or heterozygous 5/6.

Embodiments of the present invention also provide methods of identifying a decreased risk of cocaine abuse or overdose in a human subject comprising:
a.) determining, in a nucleic acid-containing sample from a human subject:
   i. the genotype of the DRD2 gene at rs2283265; and
   ii. the number of intron8 repeats in the DAT gene;
b.) identifying decreased risk of cocaine abuse or overdose if:
   i. the genotype of the DRD2 gene at rs2283265 is heterozygous G/T and the number of intron8 repeats in the DAT gene is homozygous 5/5 or heterozygous 5/6; or
   ii. the genotype of the DRD2 gene at rs2283265 is homozygous G/G and the number of intron8 repeats in the DAT gene is homozygous 6/6.

Embodiments of the present invention also provide methods as described, which further comprise determining the genotype of the DRD2 gene at rs1076560.

Embodiments of the present invention also provide methods of identifying an increased risk of cocaine abuse or overdose in a Caucasian or Hispanic human subject comprising:
a.) determining, from a biologic sample taken from a Caucasian or Hispanic human subject:
   i. the genotype of the DRD2 gene at at least one locus selected from the group consisting of rs1076560 and rs2283265; and
b.) identifying increased risk of cocaine abuse or overdose if:
   i. the genotype of the DRD2 gene at rs1076560 and/or rs2283265 is heterozygous G/T or homozygous T/T.

Embodiments of the present invention also provide methods of identifying an increased risk of cocaine abuse or overdose in a human subject comprising:
a.) determining:
   i. the genotype of the DRD2 gene at rs1076560 and rs2283265; and
b.) identifying increased risk of cocaine abuse or overdose if:
   i. the genotype of the DRD2 gene at rs1076560 and rs2283265 is heterozygous G/T or homozygous T/T.

Embodiments of the present invention also provide methods as described, which further comprise determining the genotype of the DAT gene at least one locus selected from the group consisting of: rs6347; rs27072; and rs3836790.

Embodiments of the present invention also provide methods as described, which further comprise recommending a health or legal strategy based on the results of step (b).

Embodiments of the present invention also provide methods as described, wherein determining comprises nucleic acid amplification.

Embodiments of the present invention also provide methods as described, wherein amplification comprises PCR.

Embodiments of the present invention also provide methods as described, wherein determining comprises primer extension.

Embodiments of the present invention also provide methods as described, wherein determining comprises restriction digestion.

Embodiments of the present invention also provide methods as described, wherein determining comprises sequencing.

Embodiments of the present invention also provide methods as described, wherein determining comprises SNP specific oligonucleotide hybridization.

Embodiments of the present invention also provide methods as described, wherein determining comprises a DNAse protection assay.

Embodiments of the present invention also provide methods as described, wherein said nucleic acid-containing sample is blood, sputum, saliva, mucosal scraping or tissue biopsy.

### DEFINITIONS

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described.

It must be noted that as used herein and in the appended claims, the singular forms “a”, “and”, and “the” include plural referents unless the context clearly dictates otherwise. Thus, for example, reference to “a polymorphism includes a plurality of such polymorphisms, reference to “a nucleic acid molecule” includes a plurality of such nucleic acid molecules, and reference to “the method” includes reference to one or more methods, method steps, and equivalents thereof known to those skilled in the art, and so forth.

The term “polymorphism”, as used herein, refers to a difference in the nucleotide or amino acid sequence of a given region as compared to a nucleotide or amino acid sequence in a homologous-region of another individual, in particular, a difference in the nucleotide of amino acid sequence of a given region which differs between individuals of the same species. A polymorphism is generally defined in relation to a reference sequence. Polymorphisms include single nucleotide differences, differences in sequence of more than one nucleotide, and single or multiple nucleotide insertions, inversions and deletions; as well as single amino acid differences, differences in sequence of more than one amino acid, and single or multiple amino acid insertions, inversions, and deletions.

An “allele” in relation to single nucleotide polymorphisms is the presence of a particular nucleotide at a particular genomic location. Alleles can be classified as “high risk alleles” if the presence of a particular nucleotide at a specific location is associated with an increased risk of developing a particular disease. Alleles can also be classified as “low risk alleles” if the presence of a particular nucleotide at a specific location does not appear to be associated with an increased risk of developing a particular disease.

The terms “polynucleotide” and “nucleic acid molecule” are used interchangeably herein to refer to polymeric forms of nucleotides of any length. The polynucleotides may contain deoxyribonucleotides, ribonucleotides, and/or their analogs. Nucleotides may have any three-dimensional structure, and may perform any function, known or unknown. The term “polynucleotide” includes single-, double-stranded and triple helical molecules. “Oligonucleotide” generally refers to polynucleotides of between about 5 and about 100 nucleotides of single- or double-stranded DNA. However, for the purposes of this disclosure, there is no upper limit to the length of an oligonucleotide. Oligonucleotides are also known as oligomers or oligos and may be isolated from genes, or chemically synthesized by methods known in the art.

The following are non-limiting embodiments of polynucleotides: a gene or gene fragment, exons, introns, mRNA, tRNA, rRNA, ribozymes, cDNA, recombinant polynucleotides, branched polynucleotides, plasmids, vectors, isolated DNA of any sequence, isolated RNA of any sequence, nucleic acid probes, and primers. A nucleic acid molecule may also comprise modified nucleic acid molecules, such as methylated nucleic acid molecules and nucleic acid molecule analogs. Analogs of purines and pyrimidines are known in the art. Nucleic acids may be naturally occurring, e.g. DNA or RNA, or may be synthetic analogs, as known in the art. Such analogs may be preferred for use as probes because of superior stability under assay conditions. Modifications in the native structure, including alterations in the backbone, sugars or heterocyclic bases, have been shown to increase intracellular stability and binding affinity.

In the broadest sense, as used herein, the terms “a dopamine dysregulation disorder,” refer to a condition or disease which results, directly or indirectly, from altered DRD2 or DAT activity. “Altered DRD2 or DAT activity,” as used herein, includes one or more of the following: (1) DRD2 or DAT biological activity that is higher or lower than normal DRD2 or DAT biological activity; (2) a level of DRD2 or DAT mRNA in a cell that is higher or lower than the normal level of DRD2 or DAT mRNA for that cell type; and (3) a level of DRD2 or DAT polypeptide that is higher or lower than the normal level of DRD2 or DAT polypeptide. A condition associated with DRD2 or DAT activity is also a condition or disease which is symptomatic of altered DRD2 or DAT activity. “Normal DRD2 or DAT biological activity,” “normal DRD2 or DAT mRNA levels,” and “normal DRD2 or DAT polypeptide levels” refer to DRD2 or DAT activity that is in the normal range for an individual of a given species, and which is not associated with, or give rise to, a disease condition.

The terms “determine an increased/decreased risk of a dopamine dysregulation disorder”, or “determine an increased/decreased risk of cocaine abuse or overdose,” as used herein, refers to a statistically significant increase in the probability of developing measurable characteristics of a condition associated with DRD2 or DAT activity in an individual having a particular genetic lesion(s) or polymorphism(s) compared with the probability in an individual lacking the genetic lesion or polymorphism.

Hybridization reactions can be performed under conditions of different “stringency”. Conditions that increase stringency of a hybridization reaction of widely known and published in the art. See, for example, Sambrook et al. (1989). Examples of relevant conditions include (in order of increasing stringency): incubation temperatures of 25° C., 37° C., 50° C. and 68° C.; buffer concentrations of 10×SSC, 6×SSC, 1×SSC, 0.1×SSC (where SSC is 0.15 M NaCl and 15 mM citrate buffer) and their equivalents using other buffer systems; formamide concentrations of 0%, 25%, 50%, and 75%; incubation times from 5 minutes to 24 hours; 1, 2, or more washing steps; wash incubation times of 1, 2, or 15 minutes; and wash solutions of 6×SSC, 1×SSC, 0.1×SSC, or deionized water. Examples of stringent conditions are hybridization and washing at 50° C. or higher and in 0.1×SSC (9 mM NaCl/0.9 mM sodium citrate).

Stringent conditions for both DNA/DNA and DNA/RNA hybridization are as described by Sambrook et al. Molecular Cloning, A Laboratory Manual, 2nd Ed., Cold Spring Harbor Laboratory Press, Cold Spring Harbor, N.Y., 1989. For example, see page 7.52 of Sambrook et al.

The term “binds specifically,” in the context of antibody binding, refers to high avidity and/or high affinity binding of an antibody to a specific polypeptide i.e., epitope of a polymorphic DRD2 or DAT polypeptide. Antibody binding to an epitope on a specific polymorphic DRD2 or DAT polypeptide (also referred to herein as “a polymorphic DRD2 or DAT epitope”) is preferably stronger than binding of the same antibody to any other epitope, particularly those which may be present in molecules in association with, or in the same sample, as the specific polypeptide of interest, e.g., binds more strongly to a specific DRD2 or DAT polymorphic epitope than to a different DRD2 or DAT epitope so that by adjusting binding conditions the antibody binds almost exclusively to the specific DRD2 or DAT polymorphic epitope and not to any other DRD2 or DAT epitope, and not to any other DRD2 or DAT polypeptide which does not comprise the polymorphic epitope. Antibodies which bind specifically to a polypeptide of interest may be capable of binding other polypeptides at a weak, yet detectable, level (e.g., 10% or less of the binding shown to the polypeptide of interest). Such weak binding, or background binding, is readily discernible from the specific antibody binding to the compound or polypeptide of interest, e.g. by use of appropriate controls. In general, antibodies of embodiments of the invention which bind to a specific polymorphic DRD2 or DAT polypeptide with a binding affinity of 10⁷ mole/l or more, preferably 10⁸ mole/liters or more are said to bind specifically to the specific DRD2 or DAT polymorphic polypeptide. In general, an antibody with a binding affinity of 10⁶ mole/liters or less is not useful in that it will not bind an antigen at a detectable level using conventional methodology currently used.

The terms “detectably labeled antibody,” “detectably labeled anti-polymorphic DRD2 or DAT polypeptide,” “detectably labeled anti-DRD2 or DAT polymorphic epitope,” or “detectably labeled anti-DRD2 or DAT polymorphic polypeptide fragment” refer to an antibody (or antibody fragment which retains binding specificity for a polymorphic DRD2 or DAT polypeptide or epitope), having an attached detectable label. The detectable label is normally attached by-chemical conjugation, but where the label is a polypeptide, it could alternatively be attached by genetic engineering techniques. Methods for production of detectably labeled proteins are well known in the art. Detectable labels may be selected from a variety of such labels known in the art, including, but not limited to, radioisotopes, fluorophores, paramagnetic labels, enzymes (e.g., horseradish peroxidase), or other moieties or compounds which either emit a detectable signal (e.g., radioactivity, fluorescence, color) or emit a detectable signal after exposure of the label to its substrate. Various detectable label/substrate pairs (e.g., horseradish peroxidase/diaminobenzidine, avidin/streptavidin, luciferase/luciferin)), methods for labeling antibodies, and methods for using labeled antibodies are well known in the art (see, for example, Harlow and Lane, eds. (Antibodies: A Laboratory Manual (1988) Cold Spring Harbor Laboratory Press, Cold Spring Harbor, N.Y.)).

A “biological sample” encompasses a variety of sample types obtained from an individual and can be used in a diagnostic or monitoring assay. The definition encompasses blood and other liquid samples of biological origin, solid tissue samples such as a biopsy specimen or tissue cultures or cells derived therefrom and the progeny thereof. The definition also includes samples that have been manipulated in any way after their procurement, such as by treatment with reagents, solubilization, or enrichment for certain components, such as polynucleotides. The term “biological sample” encompasses a clinical sample, and also includes cells in culture, cell supernatants, cell lysates, serum, plasma, biological fluid, and tissue samples.

As used herein, the terms “treatment”, “treating”, “heath strategy” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse affect attributable to the disease. “Treatment”, as used herein, covers any treatment of a disease in a mammal, particularly in a human, and includes: (a) preventing the disease from occurring in a subject which may be predisposed to the disease but has not yet been diagnosed as having it; (b) inhibiting the disease, i.e., arresting its development; and (c) relieving the disease, i.e., causing regression of the disease.

The terms “individual,” “subject,” and “patient,” used interchangeably herein, refer to a mammal, including, but not limited to, murines, simians, humans, mammalian farm animals, mammalian sport animals, and mammalian pets.

### BRIEF DESCRIPTIONS OF THE FIGURES

The patent or application file may contain one or more drawings executed in color and/or one or more photographs. Copies of this patent or patent application publication with color drawing(s) and/or photograph(s) will be provided by the U.S. Patent and Trademark Office upon request and payment of the necessary fees.

FIG. 1. Associations of relative risk and DAT protein Bmax activity with DRD2 rs2283265 and DAT intron8 5/6 repeat. Panel A: Odds ratios (ORs) were calculated for the minor vs. major alleles of intron8 (left panels) and rs2283265 (right panels), in the context of the reciprocal alleles of the other variant. Panel B: DAT Bmax (picomole/g) levels. The main effect for intron8 is significant in the model controlling for case status (p-value 0.032). Shown are the fitted values for that model. While the confidence intervals overlap, the overall difference between the two levels of intron8 is significant controlling for case status.

FIG. 2. Associations of relative risk and DAT protein Bmax with rs2283267 and rs6347. Odds ratios (ORs) were calculated for the minor vs. major alleles of rs6347 (left panels) and rs2283267 (right panels), in the context of the reciprocal alleles of the other variant. Top panels: DAT Bmax levels (picomole/g); bottom panels case/control ORs.

FIG. 3. Schematics of the gene loci of DRD2 and DAT. Locations of the tested variants are indicated. (intron8 VNTR=intron8 5/6-repeat; 3′VNTR=3′UTR 9/10-repeat).

### DETAILED DESCRIPTION

Numerous studies have focused on DAT and DRD2 as candidate genes influencing cognitive processes and mental disorders, including drug addiction, yielding statistically significant associations but often lacking evidence for causative relationships. The inventors have identified two intronic DRD2 SNPs bracketing exon6, rs2283265 and rs1076560, occurring in high linkage disequilibrium (LD) with each other, with minor allele frequency (MAF) of ˜18% in Caucasians. The minor alleles of both SNPs enhance exon6 inclusion, leading to a reduction in D2S, which was significantly associated with cognitive processing. Moreover, rs2283265 and rs1076560 were strongly associated with risk of severe cocaine abuse and death, in a cohort of subjects (with death due to cocaine overdose) and controls (with no drug related death) from the Miami Dowd County Brain Endowment Bank (Miami Brain Bank), with an odds ratio (OR) of approximately 3. Risk was not significant in African American subjects, suggesting the presence of epistasis involving other genes. DAT also harbors numerous polymorphisms, but evidence for true in vivo functionality is limited. Detailed in vitro studies have demonstrated that an intron8 5/6-repeat (rs3836790; MAF ˜30% in Caucasians) affects expression of a minigene construct which is modulated by cellular signaling including stimulation with cocaine.

Using allelic mRNA expression in human autopsy tissues from the substantia nigra, the inventors have shown that the intron8 5-repeat leads to lower DAT mRNA levels (although a contribution of the exon7 rs6347 cannot be excluded; rs6347 is in high LD with the 5/6-repeat). Moreover, the 3′-UTR SNP rs27072 (MAF ˜18%) was found to affect DAT expression. No evidence was obtained for a direct functional role of the 9/10 repeat in the 3′-UTR (rs28363170), frequently used in clinical association studies with variable results. Studying both DAT and DRD2 polymorphisms, Kazantseva et al. (The role of dopamine transporter (slc6a3) and dopamine d2 receptor/ankyrin repeat and kinase domain containing 1 (drd2/ankk1) gene polymorphisms in personality traits. Prog Neuropsychopharmacol Biol Psychiatry. 2011; 35:1033-1040) found that each was associated with personality traits, such as neuroticism, novelty seeking, and reward dependence, but this study was not guided by molecular genetic evidence in the selection of variants and failed to address gene-gene interactions.

The results of this study reveal a profound interaction between regulatory polymorphisms in the genes encoding the D2 dopamine receptor and the dopamine transporter, in the environmental context of cocaine abuse. These two key proteins physically bind to each other presynaptically and are thought to modulate each other's functions. The two intronic DRD2 SNPs involved in this interaction have been shown to lower formation of the D2S isoforms considered to be the predominant form presynaptically, serving as an autoreceptor and enhancing the function of DAT. Therefore, the minor allele of DRD2 rs2283265 (used here to represent both intronic SNPs, which are in high LD) is expected to permit enhanced dopamine release into the synapse. It serves as a risk factor for severe cocaine abuse and death (odds ratios between 2.3-4.1 in the present report, disregarding the DAT genotype status). Accordingly, the MAF of rs2283265 is high in White cocaine cases (>40%).

The DRD2-DAT Interaction

Surprisingly, none of the four DAT variants had significant effect on cocaine abuse/death when considered alone. Both intron8 5/6-repeat and rs27072 regulate DAT expression, and the genetic effect was found to be context-dependent. When asking whether either DAT variant affects the impact of DRD2 variants on cocaine risk, the inventors find that risk conferred by the minor allele of rs2283265 increased to 7.6 (Table 3) in subjects homozygous for the major intron8 6-repeat allele, whereas no significant risk was apparent in carriers of the minor 5-repeat allele. This interaction was highly significant, indicating that the 5-repeat was protective in the presence of the DRD2 minor rs2283265 risk allele.

The interactions between the DAT and DRD2 variants can account for the inventors' previous observation that the minor DRD2 rs2283265 allele did not seem to be a risk factor in African Americans. Here, rs2283265 MAF is low (7%) whereas the protective intron8 5-repeat variant is actually the most frequent allele (˜60%, similar for rs6437 MAF). The model calculations suggest race not to play a role if one accounts for these MAF differences.

In a mirror image, the minor DAT intron8 5-repeat turned into a risk allele (OR=2.2) (Table 3) in homozygous carriers of the main rs2283265 allele. Therefore, the risk conveyed by a single minor allele in either one gene is counterbalanced by presence of a minor allele in the other gene. This unusual interaction may stem from the postulated bell-shape curve of dopamine effects wherein maximum effect arises from an intermediate activity. This interpretation is further confounded by the profound increase in DAT activity upon chronic cocaine exposure. Nevertheless, the intron8 5/6-repeat effect has been shown to change upon cocaine exposure in vitro, while in vivo the 5-repeat appears to convey increased risk unless counterbalanced by the minor DRD2 rs2283265 allele.

DRD2-DAT Effects on DAT Protein Activity

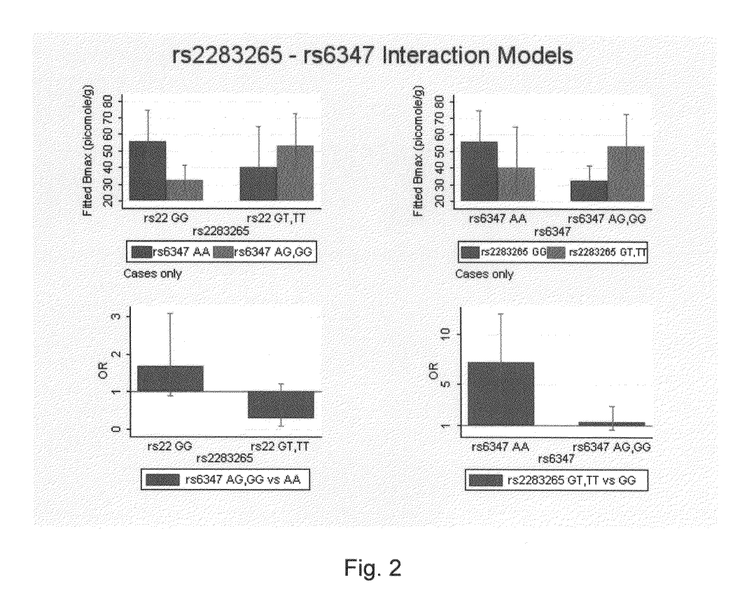

The inventors also present evidence in support of the hypothesis that the DAT and DRD2 SNPs affect the elevated DAT activity in prefrontal cortex tissues of cocaine abusers; with the intron8 5-repeat causing a significant reduction in the context of the major rs2283265 allele. However, altered DAT activity and hence inferred higher dopamine activity cannot account for the substantial risk of cocaine abuse/death attributable to the minor rs2283265 allele. Here the inventors cannot exclude the possibility that rs6437 also could play a role, showing significant interactions with rs2283265 on DAT protein activity (FIG. 2). Nevertheless, the finding of a genotype effect on DAT activity in cocaine abuser brain tissue strengthens the hypothesis that these variant may affect risk of cocaine abuse/death.

The Effect of DAT Haplotype

The inventors also considered the possibility that DAT haplotypes play a role, as a function of how the regulatory variants are phase (on the same haplotype or on opposite ones). First analysis of a 4-variant haplotype led to a two-variant haplotype with remarkably distinct distribution between cases and controls. Because of ambiguity in diplotype estimates in some individuals, haplotype estimates needed to be done separately for cases and controls. Whereas the DRD2-DAT interaction was more readily observed in Whites considering the high DRD2 rs2283265 MAF, the haplotype consisting of the minor DAT alleles of intron8 5-repeat and rs27072 in the 3′-UTR (the 22 haplotype) was more prominent in African Americans and of sufficient frequency for statistical analysis. Remarkably, estimates revealed that the 22-haplotype was probably absent in the control sample (with some minor ambiguity of haplotype estimations), whereas it was present at ˜17% frequency in the cases (one in 3-4 subjects being carriers). Calculated odds ratios depended upon the model assumptions (primarily whether distinct haplotype estimates are used for cases and controls), but ORs as high as >20 are consistent with the haplotype distributions. Only one case subject with the 22-haplotype also carried the minor allele of DRD2 rs2283265; however, because of the low MAF in African Americans, the inventors cannot discern whether rs2283265 protects against the risk conferred by the 22-haplotype. The inventors speculate that the interaction between intron8 5-repeat and the minor allele of rs27072 generates a phased haplotype—rare in the normal population—with effects on DAT activity upon cocaine abuse maximally conducive to high risk. In the inventors' previous study of DAT, the inventors have shown in vitro that the minor rs27072 allele affects translation into DAT protein in a dosage dependent manner. It is possible that cocaine-induced elevated DAT mRNA levels overcome or saturate the inhibitory effect of rs27072 at low levels, leading to a more rapid rise of DAT activity, and increasing need for cocaine administration.

Disorders or conditions of the central nervous system mean disorders affecting the spinal cord or, in particular, the brain. The term “disorder” in the sense according to the invention refers to abnormalities which are usually regarded as pathological states or functions and may reveal themselves in the form of particular signs, symptoms and/or dysfunctions. Treatments, according to embodiments of the invention, may be directed at individual disorders, i.e. abnormalities or pathological states, but it is also possible for a plurality of abnormalities, which are causally connected together where appropriate, to be combined into patterns, i.e. syndromes, which can be treated according to the invention.

The disorders which can be treated according to embodiments of the invention include in particular psychiatric and neurological disorders. These comprise in particular organic disorders, symptomatic disorders included, such as psychoses of the acute exogenous type or associated psychoses with an organic or exogenous cause, e.g. associated with metabolic disorders, infections and endocrinopathies; endogenous psychoses such as schizophrenia and schizotypal and delusional disorders; affective disorders such as depressions, mania and manic/depressive states; and combined forms of the disorders described above; neurotic and somatoform disorders, and disorders associated with stress; dissociative disorders, e.g. deficits, clouding and splitting of consciousness and personality disorders; disorders of attention and waking/sleeping behavior, such as behavioral disorders and emotional disorders starting in childhood and adolescence, e.g. hyperactivity in children, intellectual deficits, especially attention deficit disorders, disorders of memory and cognition, e.g. learning and memory impairment (impaired cognitive function), dementia, narcolepsy and sleeping disorders, e.g. restless legs syndrome; developmental disorders; anxiety states; delirium; disorders of the sex life, e.g. male impotence; eating disorders, e.g. anorexia or bulimia; addiction; and other undefined psychiatric disorders.

The disorders which can be treated according to embodiments of the invention also include parkinsonism and epilepsy and, in particular, the affective disorders associated therewith.

Addictive disorders include the psychological disorders and behavioral disorders caused by the abuse of psychotropic substances such as pharmaceuticals or drugs, and other addictive disorders such as, for example, compulsive gambling (impulse control disorders not elsewhere classified). Examples of addictive substances are: opioids (e.g. morphine, heroin, codeine); cocaine; nicotine; alcohol; substances which interact with the GABA chloride channel complex, sedatives, hypnotics or tranquilizers, for example benzodiazepines; LSD; cannabinoids; psychomotor stimulants such as 3,4-methylenedioxy-N-methylamphetamine (Ecstasy); amphetamine and amphetamine-like substances such as methylphenidate or other stimulants, including caffeine. Addictive substances requiring particular attention are opioids, cocaine, amphetamine or amphetamine-like substances, nicotine and alcohol.

CNS disorders can be drug induced, attributed to genetic predisposition, can be related to infection or trauma, or can be of unknown etiology. They comprise neuropsychiatric disorders, neurological diseases and mental illnesses, and include neurodegenerative diseases, behavioral disorders, cognitive disorders and cognitive affective disorders. There are several CNS disorders whose clinical manifestations have been attributed to CNS dysfunction (i.e., disorders resulting from inappropriate levels of neurotransmitter release, inappropriate properties of neurotransmitter receptors, and/or inappropriate interaction between neurotransmitters and neurotransmitter receptors). Several CNS disorders can be attributed to an interactive deficiency of acetylcholine, dopamine, norepinephrine and/or serotonin.

Relatively common CNS disorders include pre-senile dementia (early-onset Alzheimer's disease), senile dementia (dementia of the Alzheimer's type), micro-infarct dementia, AIDS-related dementia, vascular dementia, Creutzfeld-Jakob disease, Pick's disease, Parkinsonism including Parkinson's disease, Lewy body dementia, progressive supranuclear palsy, Huntington's chorea, tardive dyskinesia, hyperkinesia, epilepsy, mania, attention deficit disorder, anxiety, dyslexia, schizophrenia, depression, obsessive-compulsive disorders and Tourette's syndrome.

Dopamine plays a critical role in the function of the hypothalamic-pituitary-adrenal axis and in the integration of information in sensory, limbic, and motor systems.

Methods of Assaying Genotype and Repeat Copy Number of Genes

There are a large variety of techniques that can be used to assess genotype and repeat copy number of genes, and more are being discovered each day. The following is a very general discussion of a few of these techniques that can be used in accordance with the present invention.

A sample useful for practicing the methods described herein can be any biological sample of a subject, typically a human subject. The sample contains nucleic acid molecules, including portions of the gene sequences to be examined, or corresponding encoded polypeptides, depending on the particular method used. The sample can be a cell, tissue or organ sample, or can be a sample of a biological material such as a body fluid, for example blood, milk, semen, saliva. A nucleic acid sample useful for practicing the methods provided herein may be DNA or RNA. The nucleic acid sample generally is a DNA sample, suitably genomic DNA. A cDNA sample or amplification product thereof can also be used. The methods described herein can also be practiced using a sample containing polypeptides of the subject.

RFLP

Restriction Fragment Length Polymorphism (RFLP) is a technique in which different DNA sequences may be differentiated by analysis of patterns derived from cleavage of that DNA. If two sequences differ in the distance between sites of cleavage of a particular restriction endonuclease, the length of the fragments produced will differ when the DNA is digested with a restriction enzyme.

Restriction endonucleases in turn are the enzymes that cleave DNA molecules at specific nucleotide sequences depending on the particular enzyme used. Enzyme recognition sites are usually 4 to 6 base pairs in length. Generally, the shorter the recognition sequence, the greater the number of fragments generated. If molecules differ in nucleotide sequence, fragments of different sizes may be generated. The fragments can be separated by gel electrophoresis. Use of RFLP and restriction endonucleases in nucleic acid analysis requires that the nucleic acid affect cleavage of at least one restriction enzyme site.

Primer Extension

The primer and no more than three nucleoside triphosphates (NTPs) may be combined with a polymerase and the target sequence, which serves as a template for amplification. By using less than all four NTPs, it is possible to omit one or more of the polymorphic nucleotides needed for incorporation at the polymorphic site. It is important for the practice of embodiments of the present invention that the amplification be designed such that the omitted nucleotide(s) is (are) not required between the 3′ end of the primer and the target polymorphism. The primer is then extended by a nucleic acid polymerase, in a preferred embodiment by Taq polymerase. If the omitted NTP is required at the polymorphic site, the primer is extended up to the polymorphic site, at which point the polymerization ceases. However, if the omitted NTP is not required at the polymorphic site, the primer will be extended beyond the polymorphic site, creating a longer product. Detection of the extension products is based on, for example, separation by size/length which will thereby reveal which polymorphism is present.

A specific form of primer extension can be found in U.S. Ser. No. 10/407,846.

Microarrays

Oligonucleotides may be designed to hybridize directly to a target site of interest. The most common form of such analysis is where oligonucleotides are arrayed on a chip or plate in a “microarray.” Microarrays comprise a plurality of oligos spatially distributed over, and stably associated with, the surface of a substantially planar substrate, e.g., biochips. Microarrays of oligonucleotides have been developed and find use in a variety of applications, such as screening and DNA sequencing.

In gene analysis with microarrays, an array of “probe” oligonucleotides is contacted with a nucleic acid sample of interest, i.e., target. Contact is carried out under hybridization conditions and unbound nucleic acid is then removed. The resultant pattern of hybridized nucleic acid provides information regarding the genetic profile of the sample tested. Methodologies of gene analysis on microarrays are capable of providing both qualitative and quantitative information.

A variety of different arrays which may be used are known in the art. The probe molecules of the arrays which are capable of sequence specific hybridization with target nucleic acid may be polynucleotides or hybridizing analogues or mimetics thereof, including: nucleic acids in which the phosphodiester linkage has been replaced with a substitute linkage, such as phosphorothioate, methylimino, methylphosphonate, phosphoramidate, guanidine and the like; nucleic acids in which the ribose subunit has been substituted, e.g., hexose phosphodiester; peptide nucleic acids; and the like. The length of the probes will generally range from 10 to 1000 nts, where in some embodiments the probes will be oligonucleotides and usually range from 15 to 150 nts and more usually from 15 to 100 nts in length, and in other embodiments the probes will be longer, usually ranging in length from 150 to 1000 nts, where the polynucleotide probes may be single- or double-stranded, usually single-stranded, and may be PCR fragments amplified from cDNA.

The probe molecules on the surface of the substrates will correspond to selected genes being analyzed and be positioned on the array at a known location so that positive hybridization events may be correlated to expression of a particular gene in the physiological source from which the target nucleic acid sample is derived. The substrates with which the probe molecules are stably associated may be fabricated from a variety of materials, including plastics, ceramics, metals, gels, membranes, glasses, and the like. The arrays may be produced according to any convenient methodology, such as preforming the probes and then stably associating them with the surface of the support or growing the probes directly on the support. A number of different array configurations and methods for their production are known to those of skill in the art and disclosed in U.S. Pat. Nos. 5,445,934, 5,532,128, 5,556,752, 5,242,974, 5,384,261, 5,405,783, 5,412,087, 5,424,186, 5,429,807, 5,436,327, 5,472,672, 5,527,681, 5,529,756, 5,545,531, 5,554,501, 5,561,071, 5,571,639, 5,593,839, 5,599,695, 5,624,711, 5,658,734, 5,700,637, and 6,004,755.

Following hybridization, where non-hybridized labeled nucleic acid is capable of emitting a signal during the detection step, a washing step is employed where unhybridized labeled nucleic acid is removed from the support surface, generating a pattern of hybridized nucleic acid on the substrate surface. A variety of wash solutions and protocols for their use are known to those of skill in the art and may be used.

Where the label on the target nucleic acid is not directly detectable, one then contacts the array, now comprising bound target, with the other member(s) of the signal producing system that is being employed. For example, where the label on the target is biotin, one then contacts the array with streptavidin-fluorescer conjugate under conditions sufficient for binding between the specific binding member pairs to occur. Following contact, any unbound members of the signal producing system will then be removed, e.g., by washing. The specific wash conditions employed will necessarily depend on the specific nature of the signal producing system that is employed, and will be known to those of skill in the art familiar with the particular signal producing system employed.

The resultant hybridization pattern(s) of labeled nucleic acids may be visualized or detected in a variety of ways, with the particular manner of detection being chosen based on the particular label of the nucleic acid, where representative detection means include scintillation counting, autoradiography, fluorescence measurement, calorimetric measurement, light emission measurement and the like.

Prior to detection or visualization, where one desires to reduce the potential for a mismatch hybridization event to generate a false positive signal on the pattern, the array of hybridized target/probe complexes may be treated with an endonuclease under conditions sufficient such that the endonuclease degrades single stranded, but not double stranded DNA. A variety of different endonucleases are known and may be used, where such nucleases include: mung bean nuclease, S1 nuclease, and the like. Where such treatment is employed in an assay in which the target nucleic acids are not labeled with a directly detectable label, e.g., in an assay with biotinylated target nucleic acids, the endonuclease treatment will generally be performed prior to contact of the array with the other member(s) of the signal producing system, e.g., fluorescent-streptavidin conjugate. Endonuclease treatment, as described above, ensures that only end-labeled target/probe complexes having a substantially complete hybridization at the 3′ end of the probe are detected in the hybridization pattern.

Following hybridization and any washing step(s) and/or subsequent treatments, as described above, the resultant hybridization pattern is detected. In detecting or visualizing the hybridization pattern, the intensity or signal value of the label will be not only be detected but quantified, by which is meant that the signal from each spot of the hybridization will be measured and compared to a unit value corresponding the signal emitted by known number of end-labeled target nucleic acids to obtain a count or absolute value of the copy number of each end-labeled target that is hybridized to a particular spot on the array in the hybridization pattern.

Amplification of Nucleic Acids

In a particular embodiment, it may be desirable to amplify the target sequence before evaluating, for example, a single nucleotide polymorphism (SNP). Nucleic acids used as a template for amplification may be isolated from cells, tissues or other samples according to standard methodologies (Sambrook et al., 1989). In certain embodiments, analysis is performed on whole cell or tissue homogenates or biological fluid samples without substantial purification of the template nucleic acid. The nucleic acid may be genomic DNA or fractionated or whole cell RNA. Where RNA is used, it may be desired to first convert the RNA to a complementary DNA. The DNA also may be from a cloned source or synthesized in vitro.

The term “primer,” as used herein, is meant to encompass any nucleic acid that is capable of priming the synthesis of a nascent nucleic acid in a template-dependent process. Typically, primers are oligonucleotides from ten to twenty or thirty base pairs in length, but longer sequences can be employed. Primers may be provided in double-stranded or single-stranded form, although the single-stranded form is preferred.

Pairs of primers designed to selectively hybridize to nucleic acids flanking the polymorphic site are contacted with the template nucleic acid under conditions that permit selective hybridization. Depending upon the desired application, high stringency hybridization conditions may be selected that will only allow hybridization to sequences that are completely complementary to the primers. In other embodiments, hybridization may occur under reduced stringency to allow for amplification of nucleic acids containing one or more mismatches with the primer sequences. Once hybridized, the template-primer complex is contacted with one or more enzymes that facilitate template-dependent nucleic acid synthesis. Multiple rounds of amplification, also referred to as “cycles,” are conducted until a sufficient amount of amplification product is produced.

It is also possible that multiple target sequences will be amplified in a single reaction. Primers designed to expand specific sequences located in different regions of the target genome, thereby identifying different polymorphisms, would be mixed together in a single reaction mixture. The resulting amplification mixture would contain multiple amplified regions, and could be used as the source template for polymorphism detection using the methods described in this application.

A number of template dependent processes are available to amplify the oligonucleotide sequences present in a given template sample. One of the best known amplification methods is the polymerase chain reaction (referred to as PCR), which is described in detail in U.S. Pat. Nos. 4,683,195, 4,683,202 and 4,800,159, and in Innis et al., 1988.

A reverse transcriptase PCR amplification procedure may be performed when the source of nucleic acid is fractionated or whole cell RNA. Methods of reverse transcribing RNA into cDNA are well known (see Sambrook et al., 1989). Alternative methods for reverse polymerization utilize thermostable DNA polymerases. These methods are described in WO 90/07641. Polymerase chain reaction methodologies are well known in the art. Representative methods of RT-PCR are described in U.S. Pat. No. 5,882,864.

Another method for amplification is ligase chain reaction (“LCR”), disclosed in European Application No. 320 308. U.S. Pat. No. 4,883,750 describes a method similar to LCR for binding probe pairs to a target sequence. A method based on PCR and oligonucleotide ligase assay (OLA), disclosed in U.S. Pat. No. 5,912,148, may also be used.

Another ligase-mediated reaction is disclosed by Guilfoyle et al. (1997). Genomic DNA is digested with a restriction enzyme and universal linkers are then ligated onto the restriction fragments. Primers to the universal linker sequence are then used in PCR to amplify the restriction fragments. By varying the conditions of the PCR, one can specifically amplify fragments of a certain size (i.e., less than a 1000 bases).

Alternative methods for amplification of target nucleic acid sequences that may be used in the practice of embodiments of the present invention are disclosed in U.S. Pat. Nos. 5,843,650, 5,846,709, 5,846,783, 5,849,546, 5,849,497, 5,849,547, 5,858,652, 5,866,366, 5,916,776, 5,922,574, 5,928,905, 5,928,906, 5,932,451, 5,935,825, 5,939,291 and 5,942,391, GB Application No. 2 202 328, and in PCT Application No. PCT/US89/01025.

Qbeta Replicase, described in PCT Application No. PCT/US87/00880, may also be used as an amplification method in embodiments of the present invention. In this method, a replicative sequence of RNA that has a region complementary to that of a target is added to a sample in the presence of an RNA polymerase. The polymerase will copy the replicative sequence, which may then be detected.

An isothermal amplification method, in which restriction endonucleases and ligases are used to achieve the amplification of target molecules that contain nucleotide 5′-[alpha-thio]-triphosphates in one strand of a restriction site may also be useful in the amplification of nucleic acids in embodiments of the present invention (Walker et al., 1992). Strand Displacement Amplification (SDA), disclosed in U.S. Pat. No. 5,916,779, is another method of carrying out isothermal amplification of nucleic acids which involves multiple rounds of strand displacement and synthesis, i.e., nick translation.

Other nucleic acid amplification procedures include polymerization-based amplification systems (TAS), including nucleic acid sequence based amplification (NASBA) and 3SR (Kwoh et al., 1989; Gingeras et al., PCT Application WO 88/10315. European Application No. 329 822 discloses a nucleic acid amplification process involving cyclically synthesizing single-stranded RNA (ssRNA), ssDNA, and double-stranded DNA (dsDNA), which may be used in accordance with embodiments of the present invention.

PCT Application WO 89/06700 discloses a nucleic acid sequence amplification scheme based on the hybridization of a promoter region/primer sequence to a target single-stranded DNA (ssDNA) followed by polymerization of many RNA copies of the sequence. This scheme is not cyclic, i.e., new templates are not produced from the resultant RNA transcripts. Other amplification methods include “race” and “one-sided PCR” (Frohman, 1990; Ohara et al., 1989).

Another advantageous step is to prevent unincorporated NTPs from being incorporated in a subsequent primer extension reaction. Commercially available kits may be used to remove unincorporated NTPs from the amplification products. The use of shrimp alkaline phosphatase to destroy unincorporated NTPs is also a well-known strategy for this purpose.

Sequencing

DNA sequencing enables one to perform a thorough analysis of DNA because it provides the most basic information of all: the sequence of nucleotides. Maxam & Gilbert developed the first widely used sequencing methods—a “chemical cleavage protocol.” Shortly thereafter, Sanger designed a procedure similar to the natural process of DNA replication.

Sanger's method, which is also referred to as dideoxy sequencing or chain termination, is based on the use of dideoxynucleotides (ddNTP's) in addition to the normal nucleotides (NTP's) found in DNA. Dideoxynucleotides are essentially the same as nucleotides except they contain a hydrogen group on the 3′ carbon instead of a hydroxyl group (OH). These modified nucleotides, when integrated into a sequence, prevent the addition of further nucleotides. This occurs because a phosphodiester bond cannot form between the dideoxynucleotide and the next incoming nucleotide, and thus the DNA chain is terminated. Using this method, optionally coupled with amplification of the nucleic acid target, one can now rapidly sequence large numbers of target molecules, usually employing automated sequencing apparati. Such techniques are well known to those of skill in the art.

Hybridization

There are a variety of ways by which one can assess genetic profiles, and many of these rely on nucleic acid hybridization. Hybridization is defined as the ability of a nucleic acid to selectively form duplex molecules with complementary stretches of DNAs and/or RNAs. Depending on the application envisioned, one would employ varying conditions of hybridization to achieve varying degrees of selectivity of the probe or primers for the target sequence.

Typically, a probe or primer of between 13 and 100 nucleotides, preferably between 17 and 100 nucleotides in length up to 1-2 kilobases or more in length will allow the formation of a duplex molecule that is both stable and selective. Molecules having complementary sequences over contiguous stretches greater than 20 bases in length are generally preferred, to increase stability and selectivity of the hybrid molecules obtained. One will generally prefer to design nucleic acid molecules for hybridization having one or more complementary sequences of 20 to 30 nucleotides, or even longer where desired. Such fragments may be readily prepared, for example, by directly synthesizing the fragment by chemical means or by introducing selected sequences into recombinant vectors for recombinant production.

For applications requiring high selectivity, one will typically desire to employ relatively high stringency conditions to form the hybrids. For example, relatively low salt and/or high temperature conditions, such as provided by about 0.02 M to about 0.10 M NaCl at temperatures of about 50° C. to about 70° C. Such high stringency conditions tolerate little, if any, mismatch between the probe or primers and the template or target strand and would be particularly suitable for isolating specific genes or for detecting specific mRNA transcripts. It is generally appreciated that conditions can be rendered more stringent by the addition of increasing amounts of formamide.

For certain applications, for example, lower stringency conditions may be used. Under these conditions, hybridization may occur even though the sequences of the hybridizing strands are not perfectly complementary, but are mismatched at one or more positions. Conditions may be rendered less stringent by increasing salt concentration and/or decreasing temperature. For example, a medium stringency condition could be provided by about 0.1 to 0.25 M NaCl at temperatures of about 37° C. to about 55° C., while a low stringency condition could be provided by about 0.15 M to about 0.9 M salt, at temperatures ranging from about 20° C. to about 55° C. Hybridization conditions can be readily manipulated depending on the desired results.

In other embodiments, hybridization may be achieved under conditions of, for example, 50 mM Tris-HCl (pH 8.3), 75 mM KCl, 3 mM MgCl₂, 1.0 mM dithiothreitol, at temperatures between approximately 20° C. to about 37° C. Other hybridization conditions utilized could include approximately 10 mM Tris-HCl (pH 8.3), 50 mM KCl, 1.5 mM MgCl₂, at temperatures ranging from approximately 40° C. to about 72° C.

In certain embodiments, it will be advantageous to employ nucleic acids of defined sequences in combination with an appropriate means, such as a label, for determining hybridization. A wide variety of appropriate indicator means are known in the art, including fluorescent, radioactive, enzymatic or other ligands, such as avidin/biotin, which are capable of being detected. In preferred embodiments, one may desire to employ a fluorescent label or an enzyme tag such as urease, alkaline phosphatase or peroxidase, instead of radioactive or other environmentally undesirable reagents. In the case of enzyme tags, colorimetric indicator substrates are known that can be employed to provide a detection means that is visibly or spectrophotometrically detectable, to identify specific hybridization with complementary nucleic acid containing samples.

In general, it is envisioned that the probes or primers will be useful as reagents in solution hybridization, as in PCR, for detection of expression of corresponding genes, as well as in embodiments employing a solid phase. In embodiments involving a solid phase, the test DNA (or RNA) is adsorbed or otherwise affixed to a selected matrix or surface. This fixed, single-stranded nucleic acid is then subjected to hybridization with selected probes under desired conditions. The conditions selected will depend on the particular circumstances (depending, for example, on the G+C content, type of target nucleic acid, source of nucleic acid, size of hybridization probe, etc.). Optimization of hybridization conditions for the particular application of interest is well known to those of skill in the art. After washing of the hybridized molecules to remove non-specifically bound probe molecules, hybridization is detected, and/or quantified, by determining the amount of bound label. Representative solid phase hybridization methods are disclosed in U.S. Pat. Nos. 5,843,663, 5,900,481 and 5,919,626.

Detectable Labels

Various nucleic acids may be visualized in order to confirm their presence, quantity or sequence. In one embodiment, the primer is conjugated to a chromophore but may instead be radiolabeled or fluorometrically labeled. In another embodiment, the primer is conjugated to a binding partner that carries a detectable moiety, such as an antibody or biotin. In other embodiments, the primer incorporates a fluorescent dye or label. In yet other embodiments, the primer has a mass label that can be used to detect the molecule amplified. Other embodiments also contemplate the use of Taqman and Molecular Beacon probes. Alternatively, one or more of the dNTPs may be labeled with a radioisotope, a fluorophore, a chromophore, a dye or an enzyme. Also, chemicals whose properties change in the presence of DNA can be used for detection purposes. For example, the methods may involve staining of a gel with, or incorporation into the separation media, a fluorescent dye, such as ethidium bromide or Vistra Green, and visualization under an appropriate light source.

The choice of label incorporated into the products is dictated by the method used for analysis. When using capillary electrophoresis, microfluidic electrophoresis, HPLC, or LC separations, either incorporated or intercalated fluorescent dyes are used to label and detect the amplification products. Samples are detected dynamically, in that fluorescence is quantitated as a labeled species moves past the detector. If any electrophoretic method, HPLC, or LC is used for separation, products can be detected by absorption of UV light, a property inherent to DNA and therefore not requiring addition of a label. If polyacrylamide gel or slab gel electrophoresis is used, the primer for the extension reaction can be labeled with a fluorophore, a chromophore or a radioisotope, or by associated enzymatic reaction. Alternatively, if polyacrylamide gel or slab gel electrophoresis is used, one or more of the NTPs in the extension reaction can be labeled with a fluorophore, a chromophore or a radioisotope, or by associated enzymatic reaction. Enzymatic detection involves binding an enzyme to a nucleic acid, e.g., via a biotin: avidin interaction, following separation of the amplification products on a gel, then detection by chemical reaction, such as chemiluminescence generated with luminol. A fluorescent signal can be monitored dynamically. Detection with a radioisotope or enzymatic reaction requires an initial separation by gel electrophoresis, followed by transfer of DNA molecules to a solid support (blot) prior to analysis. If blots are made, they can be analyzed more than once by probing, stripping the blot, and then reprobing. If the extension products are separated using a mass spectrometer no label is required because nucleic acids are detected directly.

In the case of radioactive isotopes, tritium, ¹⁴C and ³²P are used predominantly. Among the fluorescent labels contemplated for use as conjugates include Alexa 350, Alexa 430, AMCA, BODIPY 630/650, BODIPY 650/665, BODIPY-FL, BODIPY-R6G, BODIPY-TMR, BODIPY-TRX, Cascade Blue, Cy3, Cy5,6-FAM, Fluorescein Isothiocyanate, HEX, 6-JOE, Oregon Green 488, Oregon Green 500, Oregon Green 514, Pacific Blue, REG, Rhodamine Green, Rhodamine Red, Renographin, ROX, TAMRA, TET, Tetramethylrhodamine, and/or Texas Red.

Other Methods of Detecting Nucleic Acids

Other methods of nucleic acid detection that may be used in the practice of embodiments of the instant invention are disclosed in U.S. Pat. Nos. 5,840,873, 5,843,640, 5,843,651, 5,846,708, 5,846,717, 5,846,726, 5,846,729, 5,849,487, 5,853,990, 5,853,992, 5,853,993, 5,856,092, 5,861,244, 5,863,732, 5,863,753, 5,866,331, 5,905,024, 5,910,407, 5,912,124, 5,912,145, 5,919,630, 5,925,517, 5,928,862, 5,928,869, 5,929,227, 5,932,413 and 5,935,791.

Selection and of Primers/Probes/Enzymes

The present invention relies on the use of agents that are capable of detecting single nucleotide changes in DNA. These agents generally fall into two classes—agents that hybridize to target sequences that contain the change, and agents that hybridize to target sequences that are adjacent to (e.g., upstream or 5′ to) the region of change. A third class of agents, restriction enzymes, does not hybridize, but instead cleave at a target site.

Oligonucleotide Synthesis

Oligonucleotide synthesis is well known to those of skill in the art. Various mechanisms of oligonucleotide synthesis have been disclosed in for example, U.S. Pat. Nos. 4,659,774, 4,816,571, 5,141,813, 5,264,566, 4,959,463, 5,428,148, 5,554,744, 5,574,146, 5,602,244. Basically, chemical synthesis can be achieved by the diester method, the triester method polynucleotides phosphorylase method and by solid-phase chemistry. These methods are discussed in further detail below.

Diester method. The diester method was the first to be developed to a usable state, primarily by Khorana and co-workers (Khorana, 1979). The basic step is the joining of two suitably protected deoxynucleotides to form a dideoxynucleotide containing a phosphodiester bond. The diester method is well established and has been used to synthesize DNA molecules (Khorana, 1979).

Triester method. The main difference between the diester and triester methods is the presence in the latter of an extra protecting group on the phosphate atoms of the reactants and products (Itakura et al., 1975). The phosphate protecting group is usually a chlorophenyl group, which renders the nucleotides and polynucleotide intermediates soluble in organic solvents. Therefore, purifications are done in chloroform solutions. Other improvements in the method include (i) the block coupling of trimers and larger oligomers, (ii) the extensive use of high-performance liquid chromatography for the purification of both intermediate and final products, and (iii) solid-phase synthesis.

Polynucleotide phosphorylase method. This is an enzymatic method of DNA synthesis that can be used to synthesize many useful oligodeoxynucleotides (Gillam et al., 1978). Under controlled conditions, polynucleotide phosphorylase adds predominantly a single nucleotide to a short oligodeoxynucleotide. Chromatographic purification allows the desired single adduct to be obtained. At least a trimer is required to initiate the method of adding one base at a time, a primer that must be obtained by some other method. The polynucleotide phosphorylase method works and has the advantage that the procedures involved are familiar to most biochemists.

Solid-phase methods. The technology developed for the solid-phase synthesis of polypeptides has been applied after an, it has been possible to attach the initial nucleotide to solid support material has been attached by proceeding with the stepwise addition of nucleotides. All mixing and washing steps are simplified, and the procedure becomes amenable to automation. These syntheses are now routinely carried out using automatic DNA synthesizers.

Phosphoramidite chemistry (Beaucage, 1993) has become by far the most widely used coupling chemistry for the synthesis of oligonucleotides. As is well known to those skilled in the art, phosphoramidite synthesis of oligonucleotides involves activation of nucleoside phosphoramidite monomer precursors by reaction with an activating agent to form activated intermediates, followed by sequential addition of the activated intermediates to the growing oligonucleotide chain (generally anchored at one end to a suitable solid support) to form the oligonucleotide product.

Separation of Nucleic Acids

In certain embodiments, nucleic acid products are separated by agarose, agarose-acrylamide or polyacrylamide gel electrophoresis using standard methods (Sambrook et al., 1989). Separated products may be cut out and eluted from the gel for further manipulation. Using low melting point agarose gels, the skilled artisan my remove the separated band by heating the gel, followed by extraction of the nucleic acid.

Separation of nucleic acids may also be effected by chromatographic techniques known in the art. There are many kinds of chromatography that may be used in the practice of embodiments of the present invention, including capillary adsorption, partition, ion-exchange, hydroxylapatite, molecular sieve, reverse-phase, column, paper, thin-layer, and gas chromatography as well as HPLC.

A number of the above separation platforms can be coupled to achieve separations based on two different properties. For example, some of the primers can be coupled with a moiety that allows affinity capture, and some primers remain unmodified. Modifications can include a sugar (for binding to a lectin column), a hydrophobic group (for binding to a reverse-phase column), biotin (for binding to a streptavidin column), or an antigen (for binding to an antibody column). Samples are run through an affinity chromatography column. The flow-through fraction is collected, and the bound fraction eluted (by chemical cleavage, salt elution, etc.). Each sample is then further fractionated based on a property, such as mass, to identify individual components.

Functional and Genetic Associations of Genotypes

Linkage Analysis: Diagnostic screening may be performed for polymorphisms that are genetically linked to a phenotypic variant in DRD2 or DAT activity or expression, particularly through the use of microsatellite markers or single nucleotide polymorphisms (SNP). The microsatellite or SNP polymorphism itself may not phenotypically expressed, but is linked to sequences that result in altered activity or expression. Two polymorphic variants may be in linkage disequilibrium, i.e. where alleles show non-random associations between genes even though individual loci are in Hardy-Weinberg equilibrium.

Linkage analysis may be performed alone, or in combination with direct detection of phenotypically evident polymorphisms. The use of microsatellite markers for genotyping is well documented. For examples, see Mansfield et al. (1994) Genomics 24:225-233; and Ziegle et al. (1992) Genomics 14:1026-10331. The use of SNPs for genotyping is illustrated in Underhill et al. (1996) Proc. Natl. Acad. Sci. USA 93:196-200.

Genetic linkage maps show the relative locations of specific DNA markers along a chromosome. Any inherited physical or molecular characteristic that differs among individuals and is easily detectable in the laboratory is a potential genetic marker. DNA sequence polymorphisms are useful markers because they are plentiful and easy to characterize precisely. Many such polymorphisms are located in non-coding regions and do not affect the phenotype of the organism, yet they are detectable at the DNA level and can be used as markers. Examples include restriction fragment length polymorphisms (RFLPs), which reflect sequence variations in DNA sites or differences in the length of the product, which can be cleaved by DNA restriction enzymes, microsatellite markers, which are short repeated sequences that vary in the number of repeated units, single nucleotide polymorphisms (SNPs), and the like.

The “linkage” aspect of the map is a measure of how frequently two markers are inherited together. The closer the markers are to each other physically, the less likely a recombination event will fall between and separate them. Recombination frequency thus provides an estimate of the distance between two markers. The value of the genetic map is that an inherited trait can be located on the map by following the inheritance of a DNA marker present in affected individuals, but absent in unaffected individuals, even though the molecular basis for the trait may not yet be understood.

SNPs are markers for genetic analysis of the single nucleotide polymorphism, and other simple polymorphisms, e.g. deletions, double nucleotide polymorphisms, etc. SNPs are generally biallelic systems, that is, there are two alleles that a population may have for any particular marker. This means that the information content per SNP marker is generally relatively low when compared to microsatellite markers, which may have upwards of 10 alleles. SNPs also tend to be very population-specific; a marker that is polymorphic in one population may not be very polymorphic in another.

The database at the National Institutes of Health's National Center for Biotechnology Information, particularly the Single Nucleotide Polymorphism database (NIH NCBI SNP database) may be used as a reference for the location of SNPs.

SNP markers offer a number of benefits that will make them an increasingly valuable tool. SNPs, found approximately every kilobase (see Wang et al. (1998) Science 280:1077-1082), offer the potential for generating very high density genetic maps, which will be extremely useful for developing haplotyping systems for genes or regions of interest, and because of the nature of SNPs, they may in fact be the polymorphisms associated with the disease phenotypes under study. The low mutation rate of SNPs also makes them excellent markers for studying complex genetic traits.

Substrate screening assay. Substrate screening assays are used to determine the catalytic activity of a DRD2 or DAT protein or peptide fragment on a substrate. Many suitable assays are known in the art, including the use of primary or cultured cells, genetically modified cells (e.g., where DNA encoding the DRD2 or DAT polymorphism to be studied is introduced into the cell within an artificial construct), cell-free systems, e.g. microsomal preparations or recombinantly produced enzymes in a suitable buffer, or in animals, including human clinical trials.

Typically a detectably labeled substrate is input into the assay system, and the generation of labeled triglyceride is measured over time. The choice of detection system is determined by the substrate and the specific assay parameters. Assays are conventionally run, and will include negative and positive controls, varying concentrations of substrate and enzyme, etc. Exemplary assays may be found in the literature, as described above.

Pharmacokinetic parameters. Pharmacokinetic parameters provide fundamental data for designing safe and effective dosage regimens. A drug's volume of distribution, clearance, and the derived parameter, half-life, are particularly important, as they determine the degree of fluctuation between a maximum and minimum plasma concentration during a dosage interval, the magnitude of steady state concentration and the time to reach steady state plasma concentration upon chronic dosing. Parameters derived from in vivo drug administration are useful in determining the clinical effect of a particular DRD2 or DAT genotype.

Expression assay. An assay to determine the effect of a sequence polymorphism on DRD2 or DAT expression. Expression assays may be performed in cell-free extracts, or by transforming cells with a suitable vector. Alterations in expression may occur in the basal level that is expressed in one or more cell types, or in the effect that an expression modifier has on the ability of the gene to be inhibited or induced. Expression levels of a variant alleles are compared by various methods known in the art. Methods for determining promoter or enhancer strength include quantitation of the expressed natural protein; insertion of the variant control element into a vector with a reporter gene such as galactosidase, luciferase, chloramphenicol acetyltransferase, etc. that provides for convenient quantitation; and the like.

Gel shift or electrophoretic mobility shift assay provides a simple and rapid method for detecting DNA-binding proteins (Ausubel, F. M. et al. (1989) In: Current Protocols in Molecular Biology, Vol. 2, John Wiley and Sons, New York). This method has been used widely in the study of sequence-specific DNA-binding proteins, such as transcription factors. The assay is based on the observation that complexes of protein and DNA migrate through a nondenaturing polyacrylamide gel more slowly than free DNA fragments or double-stranded oligonucleotides. The gel shift assay is performed by incubating a purified protein, or a complex mixture of proteins (such as nuclear or cell extract preparations), with an end-labeled DNA fragment containing the putative protein binding site. The reaction products are then analyzed on a nondenaturing polyacrylamide gel. The specificity of the DNA-binding protein for the putative binding site is established by competition experiments using DNA fragments or oligonucleotides containing a binding site for the protein of interest, or other unrelated DNA sequences.

Expression assays can be used to detect differences in expression of polymorphisms with respect to tissue specificity, expression level, or expression in response to exposure to various substrates, and/or timing of expression during development.

Genotyping DRD2 or DAT genotyping is performed by DNA or RNA sequence and/or hybridization analysis of any convenient sample from a patient, e.g. biopsy material, blood sample (serum, plasma, etc.), buccal cell sample, etc. A nucleic acid sample from an individual is analyzed for the presence of polymorphisms in DAT or DRD2, particularly those that affect the activity or expression of BLyS. Specific sequences of interest include any polymorphism that leads to changes in basal expression in one or more tissues, to changes in the modulation of DRD2 or DAT expression by modifiers, or alterations in DRD2 or DAT substrate specificity and/or activity.

The effect of a polymorphism in the DRD2 or DAT gene sequence on the response to a particular substrate or modifier of DRD2 or DAT is determined by in vitro or in vivo assays. Such assays may include monitoring the metabolism of a substrate during clinical trials to determine the DRD2 or DAT biological activity, specificity or expression level. Generally, in vitro assays are useful in determining the direct effect of a particular polymorphism, while clinical studies will also detect an biological phenotype that is genetically linked to a polymorphism.

The response of an individual to the substrate or modifier can then be predicted by determining the DRD2 or DAT genotype, with respect to the polymorphism. Where there is a differential distribution of a polymorphism by racial background, guidelines for drug administration can be generally tailored to a particular ethnic group.

The basal expression level in different tissue may be determined by analysis of tissue samples from individuals typed for the presence or absence of a specific polymorphism. Any convenient method may be use, e.g. ELISA, RIA, etc. for protein quantitation, northern blot or other hybridization analysis, quantitative RT-PCR, etc. for mRNA quantitation. The tissue specific expression is correlated with the genotype.

The alteration of DRD2 or DAT expression in response to a modifier is determined by administering or combining the candidate modifier with an expression system, e.g. animal, cell, in vitro transcription assay, etc. The effect of the modifier on DRD2 or DAT transcription and/or steady state mRNA levels is determined. As with the basal expression levels, tissue specific interactions are of interest. Correlations are made between the ability of an expression modifier to affect DRD2 or DAT activity, and the presence of the provided polymorphisms. A panel of different modifiers, cell types, etc. may be screened in order to determine the effect under a number of different conditions.

A DRD2 or DAT polymorphism that results in altered biological activity or specificity is determined by a variety of assays known in the art. The ligand may be tested for formation of triglyceride product in vitro, for example in defined buffer, or in cell or subcellular lysates, where the ability of a substrate to be acted on by DRD2 or DAT under physiologic conditions is determined. Where there are not significant issues of toxicity from the substrate or products(s), in vivo human trials maybe utilized, as previously described.

The genotype of an individual is determined with respect to the provided DRD2 or DAT gene polymorphisms. The genotype is useful for determining the presence of a phenotypically evident polymorphism, and for determining the linkage of a polymorphism to phenotypic change.

As discussed herein, any of a number of techniques known to those skilled in the art can be used to detect a polymorphism in a DRD2 or DAT gene, using an isolated polynucleotide as described. These include, but are not limited to, direct sequencing of the interval from affected individuals (Chadwick et al. (1996) Biotechniques 20:676-683); and hybridization with one or more probes derived from a region of a DRD2 or DAT gene, including allele-specific oligonucleotide hybridization (Wong and Senadheera (1997) Clin. Chem. 43:1857-1861). The region being detected can optionally be amplified by known techniques, including, but not limited to, a polymerase chain reaction. Other analytical techniques include, but are not limited to, single-strand conformation analysis; restriction length polymorphism (RFLP) analysis; enzymatic mismatch cleavage techniques such as glycosylase mediated polymorphism detection (Vaughan and McCarthy (1998) Nucl. Acids Res. 26:810-815); heteroduplex PCR (Deuter and Muller (1998) Hum. Mutat. 11:84-89); and fiberoptic DNA sensor array techniques (Healey et al. (1997) Anal. Biochem. 251:270-279). Automated methods of detecting polymorphisms have been developed and can be used in the methods of the present invention. See, for example, Marshall and Hodgson (1998) Nature Biotechnol 16:27-31. Other methods include, for example, PCR-RFLP. Hani et al. (1998) J. Clin. Invest. 101:521-526.

Treatment Methods

Embodiments of the present invention provide methods of treating an individual clinically diagnosed with a condition associated with DRD2 or DAT activity. The methods generally comprise analyzing a polynucleotide sample from an individual clinically diagnosed with a condition associated with DRD2 or DAT activity for the presence or absence of a DRD2 or DAT gene polymorphism. The presence of a DRD2 or DAT gene polymorphism associated with dopamine dysregulation confirms the clinical diagnosis of a condition associated with DRD2 or DAT activity. A treatment plan that is most effective for individuals clinically diagnosed as having a condition associated with DRD2 or DAT activity is then selected on the basis of the detected DRD2 or DAT polymorphism. Moreover, it is conceivable that legal decisions would be informed by the genotype, such as sentencing, institutionalization, rehabilitation, probation, and commutation decisions. Genotype information obtained as described above can be used to predict the response of the individual to a particular DRD2 or DAT substrate (e.g., activator or inhibitor of DRD2 or DAT biological activity), or modifier of DRD2 or DAT gene expression. Thus, embodiments of the invention further provide methods for predicting a patient's likelihood to respond to a drug treatment for a condition associated with DRD2 or DAT activity, comprising determining a patient's genotype in a DRD2 or DAT gene, wherein the presence of a DRD2 or DAT allele associated with a condition associated with DRD2 or DAT activity is predictive of the patient's likelihood to respond to a drug treatment for the condition. Where an expression modifier inhibits DRD2 or DAT expression, then drugs that are a DRD2 or DAT substrate will be metabolized more slowly if the modifier is co-administered. Where an expression modifier induces DRD2 or DAT expression, a co-administered substrate will typically be metabolized more rapidly. Similarly, changes in DRD2 or DAT activity will affect the metabolism of an administered drug. The pharmacokinetic effect of the interaction will depend on the metabolite that is produced, e.g. a prodrug is metabolized to an active form, a drug is metabolized to an inactive form, an environmental compound is metabolized to a toxin, etc. Consideration is given to the route of administration, drug-drug interactions, drug dosage, etc.

Thus, another aspect of the invention provides methods for tailoring an individual's prophylactic, or therapeutic treatment with DRD2 or DAT expression and/or activity modulators according to that individual's drug response genotype. Pharmacogenomics allows a clinician or physician to target prophylactic or therapeutic treatments to patients who will most benefit from the treatment and to avoid treatment of patients who will experience toxic drug-related side effects.

Agents that have a stimulatory or inhibitory effect on DRD2 or DAT expression levels or DRD2 or DAT biological activity can be administered to individuals to treat (prophylactically or therapeutically) disorders associated with DRD2 or DAT activity. Additionally, the isolated polymorphic DRD2 or DAT nucleic acid molecules of embodiments of the present invention, as well as agents, or modulators which have a stimulatory or inhibitory effect on DRD2 or DAT expression levels or DRD2 or DAT biological activity can be administered to individuals to treat a condition associated with DRD2 or DAT activity. Differences in metabolism of therapeutics can lead to severe toxicity or therapeutic failure by altering the relation between dose and blood concentration of the pharmacologically active drug. Thus, a physician or clinician may consider applying knowledge obtained in relevant pharmacogenomics studies in determining whether to administer a modulator of DRD2 or DAT expression or biological activity (“a DRD2 or DAT modulator”) as well as tailoring the dosage and/or therapeutic regimen of treatment with a DRD2 or DAT modulator.

Determination of how a given DRD2 or DAT polymorphism is predictive of a patient's likelihood of responding to a given drug treatment for a condition relating to dopamine dysregulation can be accomplished by determining the genotype of the patient in the DRD2 or DAT gene, as described above, and/or determining the effect of the drug on DRD2 or DAT gene expression, and/or determining the effect of the drug on DRD2 or DAT biological activity. Information generated from one or more of these approaches can be used to determine appropriate dosage and treatment regimens for prophylactic or therapeutic treatment an individual. This knowledge, when applied to dosing or drug selection, can avoid adverse reactions or therapeutic failure and thus enhance therapeutic or prophylactic efficiency when treating a subject with a DRD2 or DAT molecule or DRD2 or DAT modulator, such as a modulator identified by one of the exemplary screening assays described herein.

Haplotype Analysis

Haplotype analysis is known in the art, and may be conducted in the manner described in Fan H C, Wang J, Potanina A, Quake S R., Nat Biotechnol. 2011 January; 29(1): 51-7. Epub 2010 Dec. 19, Whole-genome molecular haplotyping of single cells, or as described in any of the following patents:

| PAT. NO. | Title |
|---|---|
| 7,983,848 | Computerized method and system for inferring genetic |
| | findings for a patient |
| 7,972,791 | Methods for haplotyping genomic DNA |
| 7,700,325 | Haplotype analysis |
| 7,640,113 | Methods and apparatus for complex genetics classification |
| | based on correspondence analysis and linear/quadratic analysis |
| 7,615,350 | Methods for haplotyping genomic DNA |
| 7,300,755 | Methods for haplotyping genomic DNA |
| 7,107,155 | Methods for the identification of genetic features for complex |
| | genetics classifiers |
| 6,969,589 | Methods for genomic analysis |
| 5,665,540 | Multicolor in situ hybridization methods for genetic testing |
| 5,654,148 | Multicolor in situ hybridization methods for genetic testing |

### Examples

### Example 1

### General Experimental Procedures

Postmortem Human Brain Tissues from the Miami Dowd County Brain Endowment Bank.

Prefrontal cortex (PFC) (Brodmann's area 46) and/or putamen tissue samples from cocaine abusers who died from cocaine intoxication and age-matched drug-free controls were provided by the Miami Brain Endowment Bank (University of Miami, Miami, Fla.). In this study, a total of 225 subjects were studied (126 cases (cocaine overdose deaths), 99 controls (non-drug related death)). Each group contained 86% males, 14% females; with the average age of 36 for the controls and 37 for the cases. Whites (Caucasians and Hispanics) accounted for ˜64% and non-whites for ˜36% (African Americans). Brain and blood toxicology and neuropathologic evaluations were available for each case, and most of the drug-exposure cases obtained at autopsy came from individuals who had evidence of a number of surrogate measures of chronic cocaine abuse (drug-related pathology, arrest records, and hospital and treatment admissions). Drug-free age-matched control subjects were selected from accidental or cardiac sudden deaths with negative urine screens for all common drugs, and there was no history of psychiatric disorders or licit or illicit drug use prior to death.

DNA and RNA Extraction.

DNA and RNA were extracted. cDNA was synthesized with SuperScript™ III (Life Technologies, Foster City, Calif.) using both gene-specific primers and oligo(dT).

Genotyping Methods.

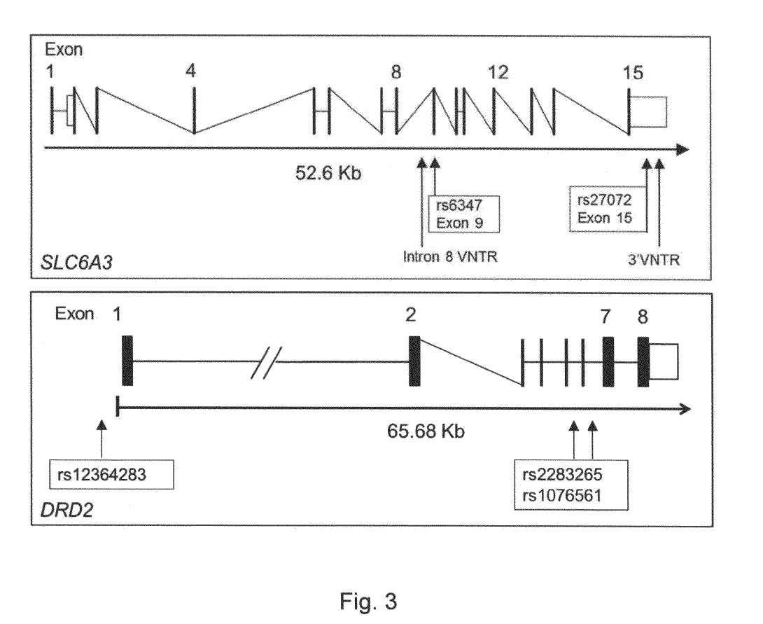

Three DRD2 and four DAT variants (SNPs and repeat polymorphisms) (Table 1) were genotyped by at least one of four methods: SNPlex (Life Technologies), SNaPshot (Life Technologies), allele-specific PCR, and a modified PCR-restriction fragment length polymorphism (RFLP) method. Repeat polymorphisms were determined with fluorescently labeled primer pairs flanking the repeats, to amplify the region with PCR, followed by analysis of fluorescently labeled fragments on an ABI 3730 capillary electrophoresis instrument (Applied Biosystems). The location of the polymorphisms in the DRD2 and DAT gene loci are shown in FIG. 3.

TABLE 1
Polymorphisms of DRD2 and DAT genotyped in this study

| | | | | MAF-All | |
|---|---|---|---|---|---|
| Marker | Gene | Location | Function | Subjects | N |
| rs1076560 | DRD2 | Intron6 | Splicing | 14.2 | 212 |
| (G > T) | | | | | |
| rs2283265 | DRD2 | Intron5 | Splicing | 13.7 | 222 |
| (G > T) | | | | | |
| rs12364283 | DRD2 | Promotor | Transcription | 5.8 | 206 |
| (A > G) | | Region | | | |
| rs3836790 | DAT | Intron8 | Regulatory | 38.3 | 223 |
| (6 > 5) | | 5/6 Repeat | | | |
| rs6347 | DAT | Exon9 | Potentially | 38.9 | 225 |
| (A > G) | | | regulatory | | |
| rs27072 | DAT | 3′ UTR | Regulatory | 14.9 | 225 |
| (C > T) | | (Exon15) | | | |
| rs28363170 | DAT | 3′UTR9/10 | Uncertain | 24.3 | 224 |
| (10 > 9) | | Repeat | evidence | | |

Statistical Analysis

Case Control Analysis.

Logistic regression was used to model the relationship between DAT and DRD2 variants with death due to cocaine overdose. Specifically, the inventors examined four DAT variants and three DRD2 variants. Single variant models, 2-variant main effects only models (ME), and 2-variant interaction models were analyzed on all subjects and among Caucasians only. The single variant analysis consisted of testing each variant under four genetic models: dominant, additive, recessive, and independent genotype. The likelihood ratio test (LRT) was used to assess the significance level of each variant under each genetic model, and for each variant the Akaike information criterion (AIC) was used to compare genetic models. Any variant with an LRT p-value smaller than 0.20 under any genetic model was then examined further in 2-variant main effect and 2-variant interaction models with each remaining variant (eliminating the fit of all pair-wise models). Fitting interaction models using the independent genotype model for each variant allowed the assessment of zero cells for each variant pair, and also the combination of genotypes by examining Wald p-values. In most cases, this resulted in modeling a dominant genetic model for each variant in the interaction models.

A small fraction of the samples were missing genotypes (see below Table 2), but with no association to any missing or non-missing observations. Therefore, available-case analysis was implemented to make proper use of all the available data for each variant. When comparing the effect sizes of 2 variants, the same sample of subjects was used. STATA 11 was used to carry out the statistical analyses.

TABLE 2
Main effect of DAT and DRD2 variants on relative risk of cocaine abuse/overdose. Each SNP was fit under four
genetic models: dominant (DOM), additive (ADD), recessive and independent genotype. The latter two scored less
well for all variants and are not shown. The likelihood ratio test was used to assess if the model fit was better
than the naïve model. AIC was calculated for each model. For rs2283265: AIC = 301.9, 300.3, 303.5,
312.2 for dominant, additive, recessive and independent genotype, respectively. Similarly, for intron8: AIC =
307.9, 306.4, 306.7, 318.4. Based purely on AIC, a dominant or additive model for rs22 would suffice; for intron8,
an additive or recessive. The recessive model for intron8 was also significant at level 0.08.

| | All Subjects | | | | | Whites Only | | | | |
|---|---|---|---|---|---|---|---|---|---|---|
| | | Dominant Model | | Additive Model | | | Dominant Model | | Additive Model | |
| Marker | N | OR | LRT p-value | OR | LRT p-value | N | OR | LRT p-value | OR | LRT p-value |
| rs1076560 | 212 | 1.96 | 0.04** | 1.8 | 0.034** | 137 | 3.3 | 0.0024*** | 2.5 | 0.0039*** |
| rs2283265 | 222 | 2.3 | 0.012** | 2.2 | 0.005*** | 142 | 4.1 | 0.0003*** | 3.3 | 0.0002*** |
| rs12364283 | 206 | 1.4 | 0.49 | 1.3 | 0.58 | 134 | 1.3 | 0.59 | 1.2 | 0.67 |
| rs3836790 | 223 | 1.5 | 0.16* | 1.4 | 0.063* | 143 | 1.2 | 0.58 | 1.2 | 0.61 |
| rs6347 | 225 | 1.2 | 0.51 | 1.2 | 0.35 | 143 | 0.99 | 0.98 | 1.0 | 0.99 |
| rs27072 | 225 | 0.94 | 0.83 | 0.96 | 0.89 | 143 | 0.5 | 0.08* | 0.5 | 0.07* |
| rs28363170 | 224 | 0.94 | 0.81 | 0.9 | 0.62 | 143 | 0.98 | 0.95 | 0.93 | 0.78 |

*p-value < 0.20 (considered for further models)
**p < 0.05;
***p < 0.01

Haplotype Analysis.

Haplotype frequencies of the four DAT variants (rs6347, intron8, rs27072, 9/10 repeat) were estimated using haplo.em (haplo.stats package from R software). Once the estimates were obtained, two widely used procedures to model the haplotype-disease association were used and results compared. The first approach is based on retrospective likelihood using STATA's haplologit command, and the second approach is based on weighted logistic regression (conducted in R). Haplologit models the joint distribution of the haplotype frequencies and regression coefficients, whereas weighted logistic regression uses a design matrix based on the posterior probabilities under a chosen genetic model. Haplologit can be more efficient than the two-step weighted logistic regression procedure for properly accounting for the variability in estimation of haplotypes, and also using the appropriate likelihood function in the case when the covariates are measured with error. However, the two-step procedure is more flexible for more complicated models, such as modeling haplotype interactions, than the current version of haplologit.

Analysis of DRD2 and DAT Variant Effects on DAT Bmax Activity

Bmax (picomole/g) of DAT activity was determined in prefrontal cortex tissues from 65 subjects of the 225 total cases and controls, measuring dopamine transport in synaptosomes, and expressed as pmole/g tissue wet weight. Of the 65 subjects, there were 32 cases and 33 controls. Males and females were divided evenly between the cases and controls (4 female cases, 5 female controls; 28 male cases, 28 male controls). Among the 45 white subjects, 20 were cases, 25 were controls, and among the 20 African Americans, 12 were cases and 8 were controls. Linear regression models were used to analyze possible predictors of Bmax (picomole/g). In response to the results of the case-control analysis, the primary hypothesis tested was if a gene-gene interaction significantly associated with case status, would also be significantly associated with Bmax (picomole/g) levels. Significance of variables was assessed by Wald p-values. Model assumptions were checked via normal quantile plots and plotting residuals versus fitted values.

### Example 2

### Single Variant Case-Control Associations

The inventors genotyped three DRD2 and four DAT variants in the Miami Cohort of 126 cases (cocaine overdose) and 99 controls (Table 1). Among these, rs1076560 and rs2283265 affect splicing of D2 mRNA, and rs1264283 increases transcription of DRD2. For DAT variants, the strongest evidence for functional relevance in vivo rests with the 3′UTR rs27072 and with rs3836790, here referred to as intron8 5/6-repeat, while an effect of rs6347 cannot be excluded. Allele frequencies in cases and controls are provided in Table 7, and LD values and MAF are in Table 8. Each of these variants was analyzed for a possible association with cocaine overdose, using 4 different genetic models (Table 2). Dominant and additive models gave significant association, while recessive and independent genotype models yielded less significant or no associations. The dominant model was adopted to accommodate less frequent variants for further analysis.

Both rs1076560 and rs2283265 (G<T) scored with significant p-values (Table 2). Since both are in high LD (Table 8), both affect splicing, and have nearly identical frequencies (in this cohort, only one subject was heterozygous for only one SNP (rs2283265)), the inventors selected rs2283265 for further analysis as it scored with slightly higher p-values Similar to previous results found by the inventors′, all subjects with the T allele present have 2.3 times the odds of cocaine overdose than those with rs2283265 GG (p-value 0.015). Analyzing only Caucasians, the OR for presence versus absence of the T allele increases from 2.3 to 4.1 (p-value 0.0007), but is estimated to be only 0.57 in African Americans (p-value 0.41). It appears that the T allele presents a large risk of cocaine overdose in Caucasians, whereas the point estimate for African Americans is not significant (see further discussion of race below).

Since rs1264283 did not appear to be significant (compromised by a low allele frequency relative to the study population), the inventors focused further analysis exclusively on rs2283265 for DRD2, considering ethnic differences in each model. It is noted that the MAF of rs1264283 is 8% in Caucasians but only 1% in African Americans (Table 8), precluding any analysis in the African American cohort.

None of the DAT variants reached significance (Table 2), while the intron8 5/6-repeat scored with p=0.06 (additive model) but only p=0.16 in Whites only. This could have been a result of the lower allele frequency of the minor 5-repeat allele in Whites (24%) compared to Non-Whites (65%) (Table 7). rs27072 scored with a marginal p-value of 0.07 in Whites (additive model, Table 2). From these results the inventors first focused on the intron8 5/6-repeat and rs2283265 for the interaction model, in comparison to all other combinations.

TABLE 7
Observed minor allele frequencies (%) for each variant studied and corresponding
sample sizes within all available subjects, all available Caucasians, and all
available African Americans. Hispanics were grouped together with Caucasians,
unless the race was marked as African American/Hispanic, in which case they were
grouped with African Americans. There was a Pacific Islander control subject
who was analyzed when using all subjects, but not when separating by race.

| | | All | | African | No. Total | No. | No. African |
|---|---|---|---|---|---|---|---|
| | | Subjects | Caucasians | Americans | Subjects | Caucasians | Americans |
| DRD2 | rs1076560 (G > T) | 14.2 | 18.3 | 6.76 | 212 | 137 | 74 |
| | rs2283265 (G > T) | 13.7 | 17.6 | 6.96 | 222 | 142 | 79 |
| | rs12364283 (A > G) | 5.83 | 8.21 | 1.41 | 206 | 134 | 71 |
| DAT | rs3836790^{a} | 38.3 | 23.8 | 65.2 | 223 | 143 | 79 |
| | rs6347 (A > G) | 38.9 | 27.6 | 59.3 | 225 | 143 | 81 |
| | rs27072 (C > T) | 14.9 | 14.7 | 15.4 | 225 | 143 | 81 |
| | rs28363170^{b} | 24.3 | 29.0 | 16.3 | 224 | 143 | 80 |

^{a}Intron8 5/6 repeat
^{b}3′UTR 9/10 repeat

TABLE 8
Pairwise LD values for DRD2 and DAT variants. The values above the main diagonal are values of R2
and below the main diagonal are D′ (i.e., for all subjects, R2 between rs1076560 and rs12364283 is
0.01, and D′ is 0.12). To save room, variants were abbreviated by the first two digits of the rs-number.
Rs3836790 is shown as “int8”, and rs2836170 is displayed as “3′UTR”.
LD MEASURES: Upper Triangle: R2; Lower Triangle: D′

| | All Subjects | | | | | Caucasians | | | | | African Americans | | | |
|---|---|---|---|---|---|---|---|---|---|---|---|---|---|---|
| | rs10 | rs12 | rs22 | rs35 | | rs10 | rs12 | rs22 | rs35 | | rs10 | rs12 | rs22 | rs35 |
| rs10 | | 0.01 | 0.94 | 0 | rs10 | | 0 | 0.93 | 0.01 | rs10 | | 0 | 1 | 0 |
| rs12 | 0.12 | | 0.01 | 0.13 | rs12 | 0.08 | | 0 | 0.15 | rs12 | 1 | | 0 | 0 |
| rs22 | 0.98 | 0.13 | | 0 | rs22 | 0.97 | 0.09 | | 0.01 | rs22 | 1 | 1 | | 0 |
| rs35 | 1 | 0.56 | 1 | | rs35 | 1 | 0.62 | 1 | | rs35 | 1 | 1 | 1 | |

| | All Subjects | | | | | Caucasians | | | | | African Americans | | | |
|---|---|---|---|---|---|---|---|---|---|---|---|---|---|---|
| | rs63 | int8 | rs27 | 3′UTR | | rs63 | int8 | rs27 | 3′UTR | | rs63 | int8 | rs27 | 3′UTR |
| rs63 | | 0.58 | 0.02 | 0.07 | rs63 | | 0.53 | 0.03 | 0.21 | rs63 | | 0.55 | 0.02 | 0.04 |
| int8 | 0.76 | | 0 | 0.08 | int8 | 0.8 | | 0.02 | 0.3 | int8 | 0.86 | | 0.01 | 0.06 |
| rs27 | 0.27 | 0.06 | | 0.04 | rs27 | 0.25 | 0.61 | | 0.07 | rs27 | 0.36 | 0.38 | | 0.01 |
| 3′UTR | 0.37 | 0.39 | 0.87 | | 3′UTR | 0.48 | 0.63 | 1 | | 3′UTR | 0.53 | 0.78 | 0.53 | |

### Example 3

### 2-Variant Interaction Models

Estimating the combined effects and interactions of DRD2 rs2283265 and DATintron8 5/6-repeat, the inventors established a significant interaction. The interaction model yielded a significant improvement over the main effect only models (LRT p-value 0.006) and was significantly better than the naïve model (p-value 0.001). To illustrate the impact of this interaction, by considering only the DRD2 SNP rs2283265 (ignoring DAT), subjects carrying the minor T allele have 2.3 times the odds of cocaine overdose than homozygous GG carriers (p=0.015; all subjects). Examining the fitted values from rs2283265 versus intron8 5/6-repeat interaction model reveals that the odds ratio of cocaine overdose associated with rs2283265 varies strongly with intron8 genotype. In carriers homozygous for the main intron8 6-repeat allele of DAT, the DRD2 rs2283265 OR increases to 7.6 (p-value 0.001) (Table 3). Conversely, in the presence of the intron8 5-repeat allele, the odds ratio for rs2283265 did not differ significantly from unity (OR 1.06, p-value 0.90) Similar results were observed in Whites only (Table 9). This result indicates a strong protective effect of the intron8 5-repeat in the presence of the rs2283265 risk allele.

The inventors then investigated whether the DAT intron8 5/6 repeat affects risk of cocaine overdosing when adjusted for DRD2 rs2283265. While the intron8 5/6 repeat considered alone does not show a significant odds ratio (Table 3), it appears to convey significant risk among homozygous carriers of the major G allele carriers at rs2283265 (OR 2.2, p-value 0.01; Table 3). This result suggests opposite effects of the intron8 5-repeat, depending upon the context of rs2283265 genotype, and vice versa. Consistent with this notion, when the rs2283265 risk (minor) allele is present, the intron8 5-repeat allele may be protective with an OR of 0.31 but showing only marginal significance (p-value 0.080; Table 3).

Similar patterns are observed with the interaction model of DRD2 rs2283265 and rs6347 A>G (LD r²=0.58, Table 8), albeit with somewhat lower significance and effect size (Table 10). The odds of cocaine overdose are significantly higher for carriers of the DRD2 rs2283265 minor T allele, but only when rs6347 is homozygous for the main A allele (OR 7.18, p-value 0.003; in Whites only, the OR is 10.6, p-value 0.003, one instance where the rs6347 scored higher than using a model with the intron8 5/6-repeat). Also, the minor rs6347 G allele lowers the odds ratio of rs2283265 (OR 1.26, p-value 0.573) Similar results for intron8 5/6 repeat and rs6347 are in part accounted for by their relatively high LD (all subjects: r²=0.58 (Table 8). None of the other 2-variant combinations showed pronounced interactions, except for combinations where rs2283265 is preplaced with rs1076560, which has a similar function and is in high LD with rs2283265.

TABLE 3
Interactions between rs22 and intron8 5/6-repeat affecting risk of
cocaine abuse and overdose. Estimated OR were obtained through linear
combinations of the coefficients using the rs2283265-intron 8 interaction
model (a dominant genetic effect for each SNP). Listed are estimated
ORs and corresponding 95% confidence intervals, Wald p-values, and
the sample sizes of the groups being compared

| | | | 95% CI | | nᵣₛ₂₂ vs. |
|---|---|---|---|---|---|
| rs2283265 | Intron8 | OR | for OR | p-value | nᵢₙₜᵣₒₙ₈ |
| GG | (56, 55)vs. | 2.2 | (1.19, 4.16) | 0.013** | 101 vs. 68 |
| | 66 | | | | |
| (GT, TT)vs. | 66 | 7.6 | (2.34, 24.7) | 0.001*** | 24 vs. 68 |
| GG | | | | | |
| GT, TT | (56, 55)vs. | 0.3 | (0.08, 1.15) | 0.080* | 28 vs. 24 |
| | 66 | | | | |
| (GT, TT)vs. | 56, 55 | 1.1 | (0.45, 2.49) | 0.901 | 28 vs. 101 |
| GG | | | | | |

*significance level 0.10;
**level 0.05;
***level 0.0001

TABLE 9
Interactions between rs2283267 and intron8 5/6-repeat affecting
risk of cocaine abuse and overdose, only in Caucasians. Estimated
OR were obtained through linear combinations of the coefficients
using the rs2283267-intron8 interaction model (a dominant genetic
effect for each SNP). Listed are ORs, Wald p-values, and the
95% confidence intervals of the ORs.

| rs2283265 | Intron8 5/6 Repeat | OR | 95% CI for OR | p-value |
|---|---|---|---|---|
| GG | (56, 55) vs. 66 | 1.50 | (0.67, 3.4) | 0.32 |
| (GT, TT)vs. GG | 66 | 7.13 | (2.16, 23.6) | 0.001 |
| GT, TT | (56, 55)vs. 66 | 0.46 | (0.11, 1.9) | 0.29 |
| (GT, TT)vs. GG | 56, 55 | 2.17 | (0.69, 6.8) | 0.19 |

TABLE 10
Interactions between DRD2 rs2283267 and DAT rs6437 affecting
risk of cocaine abuse and overdose. Estimated OR values
were obtained through linear combinations of the coefficients
using the rs2283267-rs6437 interaction model (same model
as used in TABLE 3). Listed are ORs, Wald p-values, and
the 95% confidence intervals of the ORs. Also listed are
the sample sizes of the groups being compared.

| | | | 95% CI | p- | nᵣₛ₂₂ vs. |
|---|---|---|---|---|---|
| rs2283265 | rs6347 | OR | for OR | value | nᵣₛ₆₃ |
| (GT, TT) vs. | AA | 7.2 | (1.92, 26.8) | 0.003 | 20vs68 |
| GG | | | | | |
| GG | (AG, GG)vs. | 1.7 | (0.90, 3.10) | 0.104 | 102vs68 |
| | AA | | | | |
| (GT, TT) vs. | AG, GG | 1.3 | (0.56, 2.86) | 0.573 | 32vs102 |
| GG | | | | | |
| GT, TT | (AG, GG)vs. | 0.3 | (0.07, 1.22) | 0.091 | 32vs20 |
| | AA | | | | |

### Example 4

### Impact of Race on DRD2-DATrisk Factors

Race does not appear to be a significant variable in the DAT intron8 5/6 repeat versus DRD2 rs2283265 interaction model, nor does it show evidence of acting as a confounder of the gene-gene interaction, as the interaction coefficient does not change by more than 8% (the same holds true for the interaction with rs6437 instead of the intron8 5/6-repeat). However, race could play a role when considering the model containing rs2283265 as the only variant. In this case, the OR for presence versus absence of the minor T allele increases from 2.3 to 4.1 when analyzing only Caucasians (p-value 0.0007), but the OR is estimated to be 0.57 in African Americans (p-value 0.41). Different genetic background can account for this finding, and at least in part the MAF differences between Caucasians and African Americans (Table 8). Among Caucasians, the observed MAF for intron8 5-repeat is 24%, but 65% in African Americans. Similarly, for rs6347, the MAF is 28% in Caucasians, and 59% in African Americans (60% in HapMap). If these two DAT variants were truly protective, the higher MAF accounts to a large extent for the decreased risk assignable to rs2283265 in African Americans. The interpretation of these results in African Americans is further hampered by the considerably lower MAF of rs2283265 in African Americans (7%) compared to Whites (18%).

### Example 5

### Haplotype Analysis

The inventors estimated the haplotype structure generated from the four DAT variants, shown in Table 5A. Haplotype frequencies of the four DAT variants (rs6347, intron8, rs27072, 9/10 repeat) were estimated within cases and controls, for both the Caucasians and African Americans. A substantial difference between cases and controls was observed in the African Americans; the frequency of haplotype 2221 (1=major allele, 2=minor allele) was estimated to be 15% among the 50 cases, but only 0.6% among the 31 controls (Table 5B). Of the 81 African Americans, 15 cases and 3 controls had a non-zero posterior probability for a diplotype containing haplotype 2221. The posterior probabilities were high among the cases and ranged from 0.84 to 1.0, but were only estimated to be between 0.10 and 0.16 among the controls. Only 1 of the 18 subjects, a case, has the minor T allele at rs2283265 on DRD2, which is not surprising given the allele frequency of ˜7% for African Americans (Table 7). Nevertheless, this potential risk haplotype 2221 exists primarily in the context of the major C allele at rs2283265 on DRD2. DAT Bmax (picomole/g) data were available for only 3 2221 carrier subjects, and thus the relationship with Bmax (picomole/g) cannot be statistically analyzed.

Further examination of the haplotype distribution led the inventors to focus exclusively on a 2-allele haplotype as fully representative of the suspected risk factor: the minor alleles of intron8 5/6 repeat and rs27072 (5-repeat and T allele, 22-haplotype), both variants experimentally validated as being regulatory. Haplotype distributions in Whites and African Americans are listed in Tables 5B and 5C. Again, the high allele frequency of intron8 5-repeat in African Americans directed further analysis to this group. Using only these two variants to estimate haplotype distributions in African Americans, the estimated frequency of the 22-haplotype is 18% in cases, but is estimated to be 0 in the controls (8.2e-08) (Table 5C). Because of the large difference in haplotype distributions, haplotype estimates were performed separately in cases and controls. Eleven subjects with 3 or more minor alleles had unambiguous phasing with at least 1 copy of 22-haplotype, all of whom are cases. Of 10 out of 81 African Americans with ambiguous phasing (1 minor allele at each variant), 7 were cases and 3 controls [possibly 4 controls counting the subject with intron8 genotype missing]. This results in a posterior probability for diplotype 11-22 (6C-5T) of 0.80 for the 7 ambiguous cases, but 0 for the controls.

If one ignores phasing and only considers the number of minor alleles for each African American, the OR for risk of cocaine abuse comparing 3 or more minor alleles to less than 3 alleles, is 11.6 (p-value 0.006, using exact logistic regression). If the inventors instead compare 2 or more minor alleles to less than 2, the inventors obtain an OR of 2.55 (exact p-value 0.074). This result suggests that phasing is important in conveying risk. To study the most extreme case the inventors could encounter (by imputing the most likely haplotype pair), the inventors allow the 7 ambiguous cases to have diplotype 11/22, and the 3 ambiguous controls to have diplotype 12/21 (in fact a likely scenario on the basis of haplotype estimations). If this were true, the OR from exact logistic regression comparing subjects with at least 1 copy of haplotype 22 to those without haplotype 22 would be estimated at 23 (exact p-value <0.0001). The 95% confidence interval for this OR is (3.7 to +co). The high endpoint is expected when calculating a confidence interval for the median unbiased estimate (MUE) of the odds ratio, in the situation where there is a zero cell. Important here is the low endpoint, and it is notably far from 1. The inventors can see that phasing may play an important role and leads to a hypothesis that it is not just the number of minor alleles carried by subjects, but that the intron8 5-repeat allele and rs27072 T allele are in phase on the same chromosome (22-haplotype).

Statistical methods that account for the estimation of haplotype frequencies across the entire group were used to test for a haplotype—case association among African Americans. The OR for haplotype 22 is estimated as 6.7 (p-value 0.046) under a dominant genetic model by haplologit in STATA, and is estimated as 6.29 by weighted logistic regression (p-value 0.017). Here, the ORs are comparable, but STATA results in larger standard errors, which may be more appropriate considering the added error to the model and the retrospective study design. Controlling for rs2283265 genotype (an interaction cannot be estimated here) STATA gives an OR of 8.8, p-value 0.03; whereas R gives an OR of 9.2, p-value 0.01. In neither case is the main effect of rs2283265 significant.

These varying estimates all arise from the markedly different haplotype distributions between cases and controls, in African Americans with a sufficiently high MAF of the intron8 5-repeat to permit accurate estimates in the study population. Separate haplotype estimates in cases and controls yield high and low probability, respectively, of the 22-haplotype in ambiguous subjects, while STATA and R perform haplotype estimates for the entire study population, thereby inflating the potential presence of 22 haplotypes in the control sample. Experimental phasing may be needed to address this point definitively, but by all accounts, the prevalence of the 22-haplotype in the cases vastly exceeds that in the controls, yielding high ORs in each estimate (6.0-24.0).

Table 4A, 4B, 4C. Interactions betweenDRD2 rs2283265 andDAT intron8 5/6-repeat affecting DAT protein Bmax activity.

TABLE 4A
Summary of observed DAT Bmax(picomole/g) activity.

| | Mean | SD | Min | Median | Max | N |
|---|---|---|---|---|---|---|
| Controls | 10.1 | 4.8 | 1.1 | 8.7 | 23.4 | 33 |
| Cases | 48.8 | 26.2 | 6.1 | 39.9 | 92.4 | 32 |
| Total | 29.1 | 26.9 | 1.1 | 20.3 | 92.4 | 65 |

TABLE 4B
Main effect of individual variants among White subjects. Linear
regression results for each marker (dominant coding) adjusting for
case status. The coefficients are from the models with natural log Bmax
(picomole/g) as the outcome; exp(coef) represents the ratio of Bmax
(picomole/g) between the presence and absence of the minor allele for
each variant (i.e., those with the minor allele at rs1076560 have 29%
higher Bmax(picomole/g) than those without the minor allele, adjusting
for case status). Intron8 did not score significant using all subjects
(p-value 0.25). rs6347 had the only level .1 significance among all
subjects; presence of the minor allele decreases the natural log
of Bmax(picomole/g) by 0.26 (p-value 0.07).

| | White subjects | | | | | |
|---|---|---|---|---|---|---|
| | | | | Exp | 95% CI for | |
| Marker | N | Coef (SE) | | (Coef) | Exp (coef) | p-value |
| rs1076560 | 44 | 0.25 | (.16) | 1.29 | (0.94, 1.77) | 0.12 |
| rs2283265 | 44 | 0.18 | (.16) | 1.19 | (0.86, 1.65) | 0.28 |
| rs12364283 | 44 | 0.29 | (.22) | 1.33 | (0.86, 2.07) | 0.20 |
| Intron8 5/6 Repeat | 45 | −0.32 | (.15) | 0.72 | (0.54, 0.97) | 0.032** |
| rs6347 | 45 | −0.15 | (.15) | 0.86 | (0.64, 1.17) | 0.33 |
| rs27072 | 45 | 0.11 | (.17) | 1.11 | (0.80, 1.55) | 0.58 |
| 9/10 Repeat | 45 | −0.28 | (.15) | 0.76 | (0.56, 1.03) | 0.072* |

*level 0.10;
**level 0.05 significance

TABLE 4C
Interaction model between intron8 5/6-repeat and rs2283265, controlling
for case status, among white subjects. While the interaction coefficient
was not significant for this model (p-value 0.114), we can use the fitted
values to compare to the model with only intron8 (and controlling for
case status). Ignoring rs2284265, intron8 decreases natural log Bmax
(picomole/g) by 0.32 (p-value 0.032). Using the interaction model,
among subjects who have rs2283265 GG, the intron8 5 allele decreases
natural log Bmax(picomole/g) by 0.53 (p-value 0.005).

| | | | | Exp | 95% CI for | p- |
|---|---|---|---|---|---|---|
| rs2283265 | Intron8 | Coef (SE) | | (coef) | exp (coef) | value |
| GG | (55, 56) vs. 66 | −0.53 | (0.18) | 0.59 | (0.41, 0.85) | 0.005 |
| GT, TT | (55, 56) vs. 66 | −0.03 | (0.24) | 0.97 | (0.59, 1.59) | 0.893 |
| (GT, TT) vs. | 66 | −0.05 | (0.22) | 0.96 | (0.62, 1.48) | 0.835 |
| GG | | | | | | |
| (GT, TT) vs. | (55, 56) | 0.45 | (0.21) | 1.57 | (1.02, 2.43) | 0.040 |
| GG | | | | | | |

### Example 6

### Effects of DRD2 and DAT Variants on DAT Protein Expression in Prefrontal Cortex

DAT Bmax (picomole/g) values were measured for 65 subjects using a functional transport assay with brain tissue synaptosomesas, showing a substantial increase in DAT levels in cocaine abusers (10 pmoles/g tissue in controls versus 49 in cases). Following the results of the case control analysis, the primary objective was determining the role of case status, rs2283265, rs6347 and Intron8 5/6-repeat with Bmax (picomole/g) levels. No significant SNP effect was seen without first adjusting for case status, and due to the difference in variance between the groups, both the natural log and square root transformations of Bmax (picomole/g) were used to develop models that satisfied the assumption of homoskedasticity. Finding no drastic differences between these two models, and for ease of interpretation, the natural log models are reported.

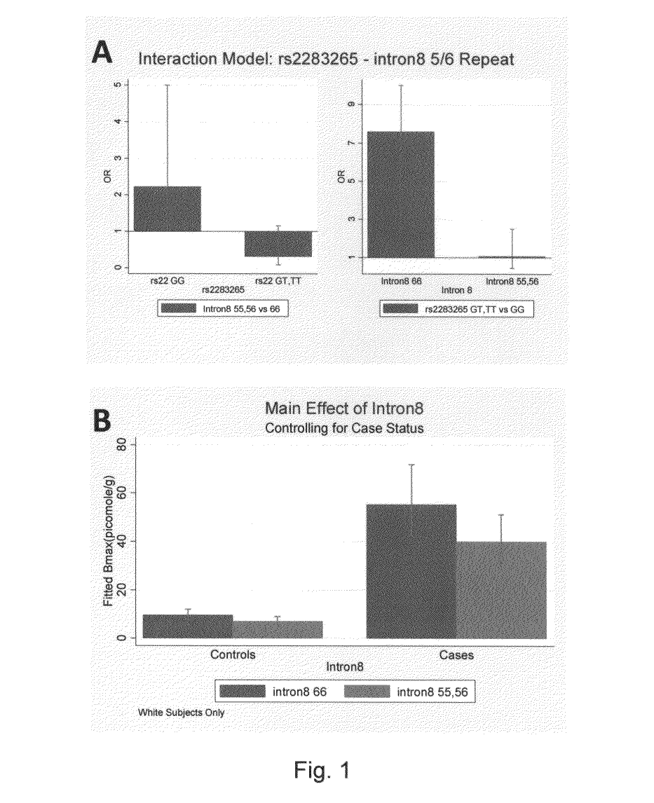

DAT intron8 5/6-repeat is significantly associated with Bmax (picomole/g) levels after adjusting for case status, in the 44 available Caucasians. From this fitted model, Caucasian carriers of the intron8 5-repeat allele have a 29% lower Bmax (picomole/g) than non-carriers, adjusting for case status (p-value 0.02) (FIG. 1). The intron8 5/6-repeat does not interact with either case status, or rs2283265, nor does it have a significant main effect when adding African Americans to the analyses where the minor allele is more abundant. None of the other variants, including rs2283265, are significantly associated with Bmax (picomole/g) controlling for either case status alone, or both case status and intron8 5/6-repeat. The effect of intron8 5/6-repeat remains significant when rs2283265 is added to the model, and rs2283265 is not a confounder.

In contrast to the findings of the case-control analysis, rs6347 effects on DAT protein activity were more pronounced or differed in some detail compared to intron8 5/6-repeat, both alone and when considering the interaction model with rs2283265 (FIG. 2). While rs6347 alone is not significant for DAT activity in Whites, controlling for case status, the rs2283265-rs6347 interaction was significant using all subjects, with a Wald p-value 0.015. Among the case subjects homozygous for the main G allele at rs2283265, those with the minor allele at rs6347 had 42% significantly lower Bmax (picomole/g) values than carriers of the minor A allele (p-value 0.002). Among case subjects with the minor rs6347 G allele present, the Bmax (picomole/g) values were 62% higher in subjects with the rs2283265 risk allele compared to subjects with rs2283265 GG (p-value 0.009). Using only the 44 Caucasians to avoid confounding effects of large MAF race differences, this interaction model was still significant (p-value 0.008). Therefore, in contrast to the findings with the intron8 5/6 repeat, the relationship between Bmax (picomole/g) and rs6347 allele status changes, depending on the rs2283265 genotype among case subjects. However, considering the rather small tissues sample number, it remains uncertain whether rs2283265 and rs6347 indeed have independent and distinct effects, or whether the effect of rs6437 on DAT activity truly differs from that of the intron8 5/6-repeat.

TABLE 5A
4-Variant haplotype distributions of DAT. Haplotypes were estimated using STATA (41), using all 4
DAT variants, in the order they occur in the gene locus (rs6347, intron8 5/6-repeat, rs27072, 3′UTR
9/10-repeat; 1 = main allele, 2 = minor allele). Because haplotypes differed between Caucasians
and African Americans, analyses were done separately for each group. Similarly, haplotypes were
estimated separately for cases and controls. For some of the case-control analyses, the frequency
estimations from the control sample were used as the initial frequencies for the likelihood model.
Within White subjects, there were 54 with ambiguous phase, 89 unambiguous. Within African Americans,
10 had ambiguous phasing, 69 had unambiguous, and 2 subjects had at least 1 genotype missing.

| Haplotype | | | | All Subjects | | | | White Subjects | | African Americans | |
|---|---|---|---|---|---|---|---|---|---|---|---|
| rs63 | int8 | rs27 | 3′UTR | Cases | Controls | Whites | AA | Cases | Controls | Cases | Controls |
| 1 | 1 | 1 | 1 | 0.39 | 0.43 | 0.40 | 0.28 | 0.51 | 0.49 | 0.27 | 0.31 |
| 1 | 1 | 1 | 2 | 0.08 | 0.08 | 0.10 | 0.00 | 0.10 | 0.10 | 0.00 | 0.00 |
| 1 | 1 | 2 | 1 | 0.05 | 0.09 | 0.09 | 0.03 | 0.06 | 0.11 | 0.02 | 0.05 |
| 1 | 2 | 1 | 1 | 0.05 | 0.04 | 0.03 | 0.07 | 0.03 | 0.02 | 0.06 | 0.07 |
| 1 | 2 | 1 | 2 | 0.01 | 0.00 | 0.01 | 0.01 | 0.02 | 0.00 | 0.01 | 0.02 |
| 1 | 2 | 2 | 1 | 0.00 | 0.00 | 0.00 | 0.01 | 0.00 | 0.00 | 0.01 | 0.00 |
| 1 | 2 | 2 | 2 | 0.00 | 0.00 | 0.00 | 0.01 | 0.00 | 0.00 | 0.01 | 0.00 |
| 2 | 1 | 1 | 1 | 0.02 | 0.02 | 0.02 | 0.01 | 0.03 | 0.01 | 0.00 | 0.04 |
| 2 | 1 | 1 | 2 | 0.01 | 0.01 | 0.01 | 0.01 | 0.01 | 0.02 | 0.01 | 0.00 |
| 2 | 1 | 2 | 1 | 0.02 | 0.04 | 0.04 | 0.00 | 0.03 | 0.04 | 0.00 | 0.00 |
| 2 | 1 | 2 | 2 | 0.00 | 0.00 | 0.00 | 0.00 | 0.00 | 0.00 | 0.00 | 0.03 |
| 2 | 2 | 1 | 1 | 0.16 | 0.13 | 0.02 | 0.33 | 0.03 | 0.01 | 0.32 | 0.36 |
| 2 | 2 | 1 | 2 | 0.13 | 0.14 | 0.16 | 0.13 | 0.15 | 0.17 | 0.13 | 0.13 |
| 2 | 2 | 2 | 1 | 0.07 | 0.00 | 0.02 | 0.11 | 0.01 | 0.02 | 0.15 | 0.01 |
| 2 | 2 | 2 | 2 | 0.00 | 0.02 | 0.00 | 0.00 | 0.00 | 0.01 | 0.00 | 0.00 |

“1” is major and “2” is minor allele. Haplotypes were estimated separately within each subgroup.

TABLE 5B
2-Variant haplotype distributions in White subjects: As none
of the 4-variant haplotypes reached significant association
with the cases, we estimated haplotypes using the EM algorithm,
selecting intron8 5/6-repeat and rs27072, both with experimentally
supported regulatory functions. Haplotypes were estimated
separately for cases and controls

| | | White | White |
|---|---|---|---|
| Haplotype | All Whites | Cases | Controls |
| 11 (6C) | 0.63 | 0.64 | 0.63 |
| 21 (5C) | 0.22 | 0.25 | 0.18 |
| 12 (6T) | 0.13 | 0.11 | 0.15 |
| 22 (5T) | 0.01 | 0.00 | 0.04 |

TABLE 5C
2-Variant haplotype distributions in African Americans

| Haplotype | All AA | AA Cases | AA Controls |
|---|---|---|---|
| 21 (5C) | 0.53 | 0.52 | 0.58 |
| 11 (6C) | 0.32 | 0.28 | 0.35 |
| 22 (5T) | 0.12 | 0.18 | 0.00 |
| 12 (6T) | 0.03 | 0.02 | 0.08 |

As shown, this haplotype, the minor alleles of intron8 5/6 repeat and rs27072 (5-repeat and T allele, 22-haplotype), was at 18% in AA cases but 0 in the controls, with an OR exceeding 20. The two minor alleles need to be in phase, i.e. reside on the same haplotype to represent the risk factor, and typically, the same individuals have the main allele at rs2283265 of DRD2. Therefore, the 5-repeat and T allele, 22-haplotype is a strong marker.

### Example 7

### Four Examples of Biomarkers Useful in Embodiments of the Present Invention

Allele combinations of the following variants: DRD2: rs1076560 (G>T; in intron6) and rs2283265 (G>T, in intron5); DAT: rs3836790 (6>5) (intron8 5/6-repeat) and rs27072 (C>T, in 3′UTR). rs1076560 and rs2283265 are in high linkage disequilibrium (LD) and have similar functions, and therefore, can serve as each others' surrogates. Another DAT SNP, rs6347 in exon8, is in high LD with rs383670 Intron8 5/6-repeat, and it can serve as a surrogate marker (although its functionality is uncertain). Therefore, the inventors used three variants (but not excluding the surrogate SNPs), each with high allele frequencies that can vary substantially between ethnic groups:

DRD2: rs2283265 (G>T; T being the minor allele)

DAT: rs383670 (intron8 5/6-repeat; the 5-repeat the minor allele) and rs27072 (C>T; T the minor allele)

Biomarker Combination 1.

Risk conferred for cocaine abuse/death by the minor T allele of rs2283265 (DRD2) was reported to be in the order of ˜3 OR (odds ratio of risk) in Caucasians (1). We now report that risk strongly depends on the allele present at the DAT intron8 5/6-repeat: in homozygous carriers of the major 6-repeat allele, the OR for the T allele of rs2283265 in DRD2 increases to 6-7 OR (the biomarker combination). In contrast, no significant risk was associated with the T allele in carriers of 1 or 2 minor 5-repeat alleles, demonstrating a strong interaction regarding dopamine dysregulation under stimulation with cocaine. No significant association with risk was observed in African Americans (AAs), for the following reasons: first, the ‘minor’ DAT intron8 5-repeat (protective in the context of rs2283265 T) has a 60% allele frequency in AAs (28% in CAUs), and second, the DRD2 T risk allele is less frequent in AAs compared to CAUs (7% versus 18%).

Biomarker Combination 2.

Risk conferred for cocaine abuse/death by the minor intron8 5-repeat allele of DAT did not reach significance, but again in the context of rs2283265 genotype, was significantly associated with cocaine abuse/death in homozygous carriers of the major G allele of rs2283265, with an OR of 2.2. This relatively lower odds ratio is nevertheless of potential importance under different external conditions, for example antipsychotics therapy, as the 5-repeat was associated with a significant lower DAT activity level in cocaine abusers, showing that it has demonstrable biochemical effects.

Biomarker Combination 3.

This variant combination is a unique DAT haplotype consisting of two minor alleles: intron8 5-repeat and rs27072 T allele (designated the 22-haplotype, 2 designating the minor allele). Risk conveyed by the 22-haplotype was studied in cocaine abusers and controls. Haplotype estimates indicate that the 22-haplotype was not detectable in the control group but has a 17% frequency in the African American cocaine abusers (1 in 3-4 would be carriers). The frequency of the 22-haplotype specifically in AAs derives from the high frequency of the 5-repeat (60%), with the rs27072 T allele being at 15%, in the entire cohort. An estimate of the potential risk factor yielded an OR of 24 (3.7-∞, the latter value owing to the absence of a 22-haplotype carrier in the controls). Hence we propose the 22-haplotype as a biomarker. This risk haplotype was much less prevalent in CAU cocaine abusers, where risk biomarker combination 1 was more prevalent. All but one carrier of the 22-haplotype (cocaine abusers) were homozygous carriers of the major G allele of rs2283265, as in biomarker combination 2 (intron8 5-repeat with rs27072 G allele). However, since the G allele has low frequency in AAs, it was not possible to determine whether the G allele might be excluded (being potentially protective). However, the 22-haplotype itself appears to be sufficient to convey high risk.

Bipolar Disorder.

The inventors also genotyped all variants listed here in a group of control subjects and bipolar disorder subjects, obtained from the Stanley Foundation, mostly CAU subjects. Here the inventors also identified the DAT 22-haplotype (minor, minor; 5,T) as a risk marker, detectable at ˜8% in the cases and 1% in the controls (Table 6). Conversely, the major, major haplotype (6,C) is overrepresented in the controls (67% versus 50%). This result establishes the 22-haplotype as a strong risk factor in CAUs for bipolar disorder in the Stanley cohort (we do not have a cohort of AAs for this indication). A further analysis of the DAT 22-haplotype interaction with the DRD2 rs2283265 reveals that again the risk 22-haplotype is associated with the main G allele of rs2283265 (in 8 subjects carrying the 22-haplotype who are also homozygous GG—yielding unambiguous rs2283265 assignment)—similar to biomarker combination 2. It is noted however, that the Stanley tissue cohort of deceased bipolar subjects contains a large portion of suicide cases, so that the 22-haplotype association could also have resulted from this sub-group of bipolar patients.

TABLE 6
Bipolar disorder data. Two-SNP DAT haplotypes (intron8 5/6
repeat and rs27-72 C > T) in the Stanley bipolar subjects
compared to controls, determined by logistic regression. The
5, T (minor, minor) haplotype is the ‘22-haplotype’, whereas
the 6, C (major, major) haplotype is the ‘11-haplotype.

| | Cases | Controls | Univariate |
|---|---|---|---|
| Haplotype | n = 48 | n = 59 | Fit |
| 5, C | 23% | 18% | 0.37 |
| 5, T | 8% | 1% | 0.001 |
| 6, C | 50% | 67% | 0.006 |
| 6, T | 16% | 12% | 0.39 |

== Logistic Regression Statistics ==

| | |
|---|---|
| Response Variable: | Case Control |
| Regression Likelihood: | −66.42 |
| Null Model Likelihood: | −73.60 |
| Sample Size: | 107 |
| chisq: | 14.37 |
| P-Value: | 0.006 |
| Regression df: | 4 |
| Residual df: | 102 |
| Total df: | 106 |

Selected Markers:
DAT in8 VNTR
DAT rs27072

Evolutionary consideration of the DAT 22-haplotype, and the need for experimental phasing of the 22-haplotype. Given the high allele frequency of both intron8 5/6-repeat (particularly in AAs) and rs27072, the extremely low 22-haplotype frequency in control AAs and CAUs suggests a strong negative selection, as these two variants are in low LD and located >20 kb apart in the genome. Therefore, one would expect a random crossing-over event to occur in the germ-line at a rate of ˜1:10,000, and 22-haplotype frequencies should rapidly equilibrate at 15-20% in AAs, which is clearly not the case (<1% in the AA controls). Negative selection pressure against 22-haplotype accumulation may be viewed as an argument strengthening the assumption of deleterious effects in humans. On the other hand, in the general population, subjects heterozygous for both the intron8 5/6-repeat and rs27072 have ambiguous phasing, preventing unequivocal haplotype assignments in single subjects (phasing in the subjects is often unambiguous because of the high haplotype frequency). Therefore, the 22-haplotype described under ‘biomarker combination 3’ must include a method for phasing haplotypes across the >20 kb distance between them—achievable for example with isolation of single DNA molecules spanning the entire region, and genotyping for each molecule separately.

While the invention has been described with reference to various and preferred embodiments, it should be understood by those skilled in the art that various changes may be made and equivalents may be substituted for elements thereof without departing from the essential scope of the invention. In addition, many modifications may be made to adapt a particular situation or material to the teachings of the invention without departing from the essential scope thereof. Therefore, it is intended that the invention not be limited to the particular embodiment disclosed herein contemplated for carrying out this invention, but that the invention will include all embodiments falling within the scope of the claims.

1. A method of identifying an increased risk of a dopamine dysregulation disorder in a human subject comprising:
a.) determining, in a nucleic acid-containing sample from a human subject:
i. the genotype of the DRD2 gene at rs2283265; and
ii. the number of intron8 repeats in the DAT gene;
b.) identifying increased risk of a dopamine dysregulation disorder if:
i. the genotype of the DRD2 gene at rs2283265 is heterozygous G/T or homozygous T/T and the number of intron8 repeats in the DAT gene is homozygous 6/6; or
ii. the genotype of the DRD2 gene at rs2283265 is homozygous G/G and the number of intron8 repeats in the DAT gene is homozygous 5/5 or heterozygous 5/6.

2. A method of identifying a decreased risk of a dopamine dysregulation disorder in a human subject comprising:
a.) determining, in a nucleic acid-containing sample from a human subject:
i. the genotype of the DRD2 gene at rs2283265; and
ii. the number of intron8 repeats in the DAT gene;
b.) identifying decreased risk of a dopamine dysregulation disorder if:
i. the genotype of the DRD2 gene at rs2283265 is homozygous T/T and the number of intron8 repeats in the DAT gene is homozygous 5/5 or heterozygous 5/6.

3. A method of identifying a decreased risk of a dopamine dysregulation disorder in a human subject comprising:
a.) determining, in a nucleic acid-containing sample from a human subject:
i. the genotype of the DRD2 gene at rs2283265; and
ii. the number of intron8 repeats in the DAT gene;
b.) identifying decreased risk of a dopamine dysregulation disorder if:
i. the genotype of the DRD2 gene at rs2283265 is heterozygous G/T and the number of intron8 repeats in the DAT gene is homozygous 5/5 or heterozygous 5/6; or
ii. the genotype of the DRD2 gene at rs2283265 is homozygous G/G and the number of intron8 repeats in the DAT gene is homozygous 6/6.

4. A method of claim 1, which further comprises determining the genotype of the DRD2 gene at rs1076560.

5. A method of identifying an increased risk of a dopamine dysregulation disorder in a Caucasian or Hispanic human subject comprising:
a.) determining, in a nucleic acid-containing sample from Caucasian or Hispanic human subject:
i. the genotype of the DRD2 gene at at least one locus selected from the group consisting of rs1076560 and rs2283265; and
b.) identifying increased risk of a dopamine dysregulation disorder if:
i. the genotype of the DRD2 gene at rs1076560 and/or rs2283265 is heterozygous G/T or homozygous T/T.

6. A method of identifying an increased risk of a dopamine dysregulation disorder in a human subject comprising:
a.) determining, in a nucleic acid-containing sample from a human subject:
i. the genotype of the DRD2 gene at rs1076560 and rs2283265; and
b.) identifying increased risk of a dopamine dysregulation disorder if:
i. the genotype of the DRD2 gene at rs1076560 and rs2283265 is heterozygous G/T or homozygous T/T.

7. A method of claim 1, which further comprises determining the genotype of the DAT gene at least one locus selected from the group consisting of: rs6347; rs27072; and rs3836790.

8. A method of claim 1, further comprising recommending a health or legal strategy based on the results of step (b).

9. A method of claim 1, wherein determining comprises nucleic acid amplification.

10. A method of claim 1, wherein amplification comprises PCR.

11. A method of claim 1, wherein determining comprises primer extension.

12. A method of claim 1, wherein determining comprises restriction digestion.

13. A method of claim 1, wherein determining comprises sequencing.

14. A method of claim 1, wherein determining comprises SNP specific oligonucleotide hybridization.

15. A method of claim 1, wherein determining comprises a DNAse protection assay.

16. A method of claim 1, wherein said sample is blood, sputum, saliva, mucosal scraping or tissue biopsy.

17. A method of claim 1, wherein the dopamine dysregulation disorder is selected from the group consisting of: pre-senile dementia (early-onset Alzheimer's disease), senile dementia (dementia of the Alzheimer's type), micro-infarct dementia, AIDS-related dementia, vascular dementia, Parkinsonism including Parkinson's disease, Lewy body dementia, progressive supranuclear palsy, Huntington's chorea, tardive dyskinesia, hyperkinesia, epilepsy, mania, attention deficit disorder, anxiety, dyslexia, schizophrenia, depression, obsessive-compulsive disorders, alcoholism, obesity, pathological gambling, attention deficit hyperactivity disorder, Tourette syndrome, cocaine dependence, nicotine dependence, polysubstance abuse, methamphetamine abuse, morphine abuse, morphine-analogue abuse, prescription drug abuse, illegal drug abuse, and addiction disorders.

18. A method of identifying an increased risk of cocaine abuse or overdose in a human subject comprising:
a.) determining, in a nucleic acid-containing sample from a human subject:
i. the genotype of the DRD2 gene at rs2283265; and
ii. the number of intron8 repeats in the DAT gene;
b.) identifying increased risk of cocaine abuse or overdose if:
i. the genotype of the DRD2 gene at rs2283265 is heterozygous G/T or homozygous T/T and the number of intron8 repeats in the DAT gene is homozygous 6/6; or
ii. the genotype of the DRD2 gene at rs2283265 is homozygous G/G and the number of intron8 repeats in the DAT gene is homozygous 5/5 or heterozygous 5/6.

19. A method of identifying a decreased risk of cocaine abuse or overdose in a human subject comprising:
a.) determining, in a nucleic acid-containing sample from a human subject:
i. the genotype of the DRD2 gene at rs2283265; and
ii. the number of intron8 repeats in the DAT gene;
b.) identifying decreased risk of developing a dopamine dysregulation disorder if:
i. the genotype of the DRD2 gene at rs2283265 is homozygous T/T and the number of intron8 repeats in the DAT gene is homozygous 5/5 or heterozygous 5/6.

20. A method of identifying a decreased risk of cocaine abuse or overdose in a human subject comprising:
a.) determining, in a nucleic acid-containing sample from a human subject:
i. the genotype of the DRD2 gene at rs2283265; and
i. the number of intron8 repeats in the DAT gene;
b.) identifying decreased risk of cocaine abuse or overdose if:
i. the genotype of the DRD2 gene at rs2283265 is heterozygous G/T and the number of intron8 repeats in the DAT gene is homozygous 5/5 or heterozygous 5/6; or
ii. the genotype of the DRD2 gene at rs2283265 is homozygous G/G and the number of intron8 repeats in the DAT gene is homozygous 6/6.

21. A method of claim 18, which further comprises determining the genotype of the DRD2 gene at rs1076560.

22. A method of identifying an increased risk of cocaine abuse or overdose in a Caucasian or Hispanic human subject comprising:
a.) determining, in a nucleic acid-containing sample from a Caucasian or Hispanic human subject:
i. the genotype of the DRD2 gene at at least one locus selected from the group consisting of rs1076560 and rs2283265; and
b.) identifying increased risk of cocaine abuse or overdose if:
i. the genotype of the DRD2 gene at rs1076560 and/or rs2283265 is heterozygous G/T or homozygous T/T.

23. A method of identifying an increased risk of cocaine abuse or overdose in a human subject comprising:
a.) determining, in a nucleic acid-containing sample from a human subject:
i. the genotype of the DRD2 gene at rs1076560 and rs2283265; and
b.) identifying increased risk of cocaine abuse or overdose if:
i. the genotype of the DRD2 gene at rs1076560 and rs2283265 is heterozygous G/T or homozygous T/T.

24. A method of claim 18, which further comprises determining the genotype of the DAT gene at least one locus selected from the group consisting of: rs6347; rs27072; and rs3836790.

25. A method of claim 18, further comprising recommending a health or legal strategy based on the results of step (b).

26. A method of claim 18, wherein determining comprises nucleic acid amplification.

27. A method of claim 18, wherein amplification comprises PCR.

28. A method of claim 18, wherein determining comprises primer extension.

29. A method of claim 18, wherein determining comprises restriction digestion.

30. A method of claim 18, wherein determining comprises sequencing.

31. A method of claim 18, wherein determining comprises SNP specific oligonucleotide hybridization.

32. A method of claim 18, wherein determining comprises a DNAse protection assay.

33. A method of claim 18, wherein said sample is blood, sputum, saliva, mucosal scraping or tissue biopsy.

34. A method of identifying an increased risk of a dopamine dysregulation disorder in a human subject comprising:
a.) determining, in a nucleic acid-containing sample from a human subject:
i. the genotype of the DRD2 gene at rs27072; and
ii. the number of intron8 repeats in the DAT gene; and
iii. the phasing of the alleles in rs27072 and intron8 repeat, generating haplotypes
b.) identifying increased risk of a dopamine dysregulation disorder if:
i. the T allele (minor) of the rs27072 and the intron8 5-repeat reside on the same haplotype, a single such haplotype in a subject being sufficient to convey risk;
ii. the genotype of the DRD2 gene at rs27072 is homozygous T/T and/or the number of intron8 repeats in the DAT gene is homozygous 5/5 (in which case the haplotype phasing is unambiguous for the presence of the T allele (minor) of the rs27072 and the intron8 5-repeat haplotype;
iii. the genotype of the DRD2 gene at rs27072 is heterozygous G/T and the intron8 repeat is heterozygous 5/6 (in which case the haplotype phasing is ambiguous), and it is experimentally determined that the T allele and the 5-repeat are on the same haplotype in phase.

35. A method of identifying a increased risk of a cocaine abuse or overdose in a human subject comprising:
a.) determining, in a nucleic acid-containing sample from a human subject:
i. the genotype of the DRD2 gene at rs27072; and
ii. the number of intron8 repeats in the DAT gene;
iii. the phasing of the T alleles in rs27072 with the 5-repeat in intron8 into the risk haplotype, either computationally or experimentally where ambiguous
b.) identifying increased risk of a cocaine abuse or overdose if:
i. the T allele (minor) of the rs27072 and the intron8 5-repeat reside on the same haplotype, a single such haplotype in a subject being sufficient to convey risk;
ii. the genotype of the DRD2 gene at rs27072 is homozygous T/T and/or the number of intron8 repeats in the DAT gene is homozygous 5/5 (in which case the haplotype phasing is unambiguous for the presence of the T allele (minor) of the rs27072 and the intron8 5-repeat haplotype;
iii. the genotype of the DRD2 gene at rs27072 is heterozygous G/T and the intron8 repeat is heterozygous 5/6 (in which case the haplotype phasing is ambiguous), and it is experimentally determined that the T allele and the 5-repeat are on the same haplotype in phase.

36. A method of identifying a increased risk of bipolar disorder in a human subject comprising:
a.) determining, in a nucleic acid-containing sample from a human subject:
i. the genotype of the DRD2 gene at rs27072; and
ii. the number of intron8 repeats in the DAT gene;
b.) identifying increased risk of bipolar disorder if:
i. the T allele (minor) of the rs27072 and the intron8 5-repeat reside on the same haplotype, a single such haplotype in a subject being sufficient to convey risk;
ii. the genotype of the DRD2 gene at rs27072 is homozygous T/T and/or the number of intron8 repeats in the DAT gene is homozygous 5/5 (in which case the haplotype phasing is unambiguous for the presence of the T allele (minor) of the rs27072 and the intron8 5-repeat haplotype;
iii. the genotype of the DRD2 gene at rs27072 is heterozygous G/T and the intron8 repeat is heterozygous 5/6 (in which case the haplotype phasing is ambiguous), and it is experimentally determined that the T allele and the 5-repeat are on the same haplotype in phase.

37. A method of claim 34, which further comprises determining the genotype of the DAT gene at least one locus selected from the group consisting of: rs1076560 and/or rs2283265; rs6347; and rs3836790.

38. A method of claim 34, further comprising recommending a health or legal strategy based on the results of step (b).

39. A method of claim 34, wherein determining comprises nucleic acid amplification.

40. A method of claim 34, wherein amplification comprises PCR.

41. A method of claim 34, wherein determining comprises primer extension.

42. A method of claim 34, wherein determining comprises restriction digestion.

43. A method of claim 34, wherein determining comprises sequencing.

44. A method of claim 34, wherein determining comprises SNP specific oligonucleotide hybridization.

45. A method of claim 34, wherein determining comprises a DNAse protection assay.

46. A method of claim 34, wherein said sample is blood, sputum, saliva, mucosal scraping or tissue biopsy.
